(19) United States
(12) Patent Application Publication
(10) Pub. No.: US 2018/0168190 A1
(43) Pub. Date: Jun. 21, 2018

(54) EUGLENA DERIVED ANIMAL FEED COMPOSITION

(71) Applicant: ALGAEON, INC., Indianapolis, IN (US)

(72) Inventors: Brad M. COX, Fishers, IN (US); Derek E. JAMROG, Indianapolis, IN (US)

(21) Appl. No.: 15/898,722

(22) Filed: Feb. 19, 2018

Related U.S. Application Data
(63) Continuation-in-part of application No. 15/177,383, filed on Jun. 9, 2016, now Pat. No. 9,901,606.

(51) Int. Cl. A23K 10/12; A23K 20/174; A61K 31/716; A61K 36/02

(52) U.S. Cl. CPC A23K 10/12; A61K 36/02; A61K 31/716; A23K 20/174

(57) ABSTRACT

An animal feed composition includes a feed component made from corn, soy, corn or soy derivatives or byproducts, or grains and an added whole cell Euglena biomass, including beta-1,3-glucan having at least about 90 percent linear, unbranched beta-1,3-glucan polysaccharide polymers with an average molecular weight of about 1.2 to 580 kilodaltons (kDa). Beta-glucan polymer chains have a polymer length of about 7.0 to 3,400 glucose monomers. The whole cell Euglena biomass has at least 30 percent beta-1,3-glucan and residual media remaining from a heterotrophic fermentation process and is about 0.0001 to 0.0124 percent of the composition. It is possible to add a Euglena lysate.

### PRIORITY APPLICATION(S)

This is a continuation-in-part application of U.S. application Ser. No. 15/177,383 filed Jun. 9, 2016, the disclosure which is hereby incorporated by reference in its entirety.

### FIELD OF THE INVENTION

The present invention relates to the field of genus Euglena organisms, and more particularly, this invention relates to a Euglena lysate composition.

### BACKGROUND OF THE INVENTION

Beta-glucans are a group of β-D-glucose polysaccharides that are produced by bacteria, yeast, algae, fungi, and in cereals. The properties of the beta-glucans depend on the source, for example, whether from bacteria, algae, yeast or other sources. Usually beta-glucans form a linear backbone with 1,3 beta-glycosidic bonds. It is known that incorporating beta-glucans within a human or animal diet has advantages. Some beta-glucans may aid in immune modulation and decrease the levels of saturated fats and reduce the risk of heart disease. It is also known that different types of beta-glucans have different effects on human physiology. For example, cereal beta-glucans may affect blood glucose regulation in those having hypercholesterolemia, while mushroom beta-glucans may act as biological response modifiers on the immune system. In some cases, it has been found that yeast beta-glucans may decrease levels of IL4 and IL5 cytokines that relate to allergic rhinitis and increase the levels of IL12.

It has also been determined that Euglena gracilis biomass containing paramylon (beta-1,3-glucan) can enhance the immune function of an individual. Paramylon is a linear (unbranched) beta-1,3-glucan polysaccharide polymer with a high molecular weight. This unbranched polymer is distinct from the other beta-glucans such as the branched beta-(1,3; 1,6)-glucans from the cell walls of yeast and cereals, for example, oats and barley; and branched beta-1,3-glucans with beta-(1,4)-glycosidic bonds forming polysaccharide side chains such as found in mushrooms.

An advantage of the beta-glucan from Euglena is that it lacks beta-(1,6), beta(1,4), and beta(1,2) bonds and any side branching structures. As a molecule and similar to some other glucans that have branching, this linear beta-glucan is insoluble and believed to be homogenous and have higher combined localization and binding affinities for receptors involved in immune response. Paramylon may be obtained from Euglena gracilis algae, which is a protist organism, and a member of the micro-algae division euglenophyceae within the euglenales family and includes many different autotrophic and heterotrophic species which can also produce paramylon. These protists can be found in enriched fresh waters, such as shallow water rivers, lakes and ponds. Paramylon is an energy-storage compound for the Euglenoids and comparable to the starch or oil and fats in other algae. Paramylon is produced in the pyrenoids and stored as granules in the cytoplasm. The paramylon granules in Euglena gracilis are oblong and about 0.5-2 micrometers (um) in diameter. Euglena gracilis stock cultures are usually maintained in controlled laboratory conditions and used as an initial inoculum source. Euglena gracilis may be manufactured axenically in closed, sterilizable bioreactors. The Euglena gracilis inoculum may be transferred to seed bioreactors to accumulate larger amounts of biomass and then passaged up to larger bioreactors as needed.

It is desirable to scale-up production of such linear, unbranched beta-1,3-glucan from genus Euglena organisms, and more particularly, Euglena gracilis using improved fermentation techniques. Euglena gracilis derived beta-glucan may confer advantageous properties for human and other animal health, including enhanced immune response and other health promoting properties. It is desirable to form a beta-glucan composition that will have enhanced properties for improved immune modulation and other uses.

### SUMMARY OF THE INVENTION

This summary is provided to introduce a selection of concepts that are further described below in the Detailed Description. This summary is not intended to identify key or essential features of the claimed subject matter, nor is it intended to be used as an aid in limiting the scope of the claimed subject matter.

An animal feed composition comprises a feed component comprising one or more of corn, soy, corn or soy derivatives or byproducts, or grains. An added whole cell Euglena biomass, includes beta-1,3-glucan comprising at least about 90 percent linear, unbranched beta-1,3-glucan polysaccharide polymers having an average molecular weight of about 1.2 to 580 kilodaltons (kDa) and beta-glucan polymer chains having a polymer length of about 7.0 to 3,400 glucose monomers. The whole cell Euglena biomass comprises at least 30 percent beta-1,3-glucan and includes residual media remaining from a heterotrophic fermentation process that produced the whole cell Euglena biomass. The added whole cell Euglena biomass and residual media are about 0.0001 to 0.0124 percent w/w of the composition.

In yet another example, the added whole cell Euglena biomass and residual media may be about 0.001 to 0.01 percent w/w of the composition. The composition may further comprise an added immune response inducing component comprising one or more of vitamin C, Echinacea, aloe, golden seal, ginseng, garlic, bell peppers, ginger, tumeric, gingko biloba, cat's claw, ganoderma, astragalus, humic or fulvic acids, resveratrol or other polyphenols, broccoli, spinach, yogurt, almonds, honey, green tea, papaya, kiwi, poultry, shrimp, sunflower, vitamin D, mushrooms, pumpkin, cinnamon, parsnips, grapes, sweet potatoes, milk, orange juice, rice, carotenoids, figs, glutamine, arginine, an omega-3 fatty acid, vitamin A, vitamin E, selenium, zinc, or a probiotic.

The one or more of corn, soy, corn or soy derivatives or byproducts, or grains may comprise about 40% to 95% w/w of the composition and the feed component may further comprise protein in an amount of about 15% to 30% w/w of the composition and lysine in an amount of about 0.60% to 2.0% w/w of the composition. The residual media remaining from a heterotrophic fermentation process may comprise about 10 percent of an initial fermentation concentration.

In another example, the animal feed composition may comprise a feed component comprising one or more of corn, soy, corn or soy derivatives or byproducts, or grains and an added Euglena biomass lysate having an average particle size of about 2.0 to 500 micrometers and comprising cellular components, including beta-1,3-glucan comprising at least about 90 percent linear, unbranched beta-1,3-glucan polysaccharide polymers having an average molecular weight of about 1.2 to 580 kilodaltons (kDa) and beta-glucan polymer chains having a polymer length of about 7.0 to 3,400 glucose monomers. The Euglena lysate comprises at least 30 percent beta-1,3-glucan and includes residual media remaining from a heterotrophic fermentation process that produced a Euglena biomass and the Euglena lysate. The added Euglena lysate and residual media are about 0.0001 to 0.0124 percent w/w of the composition.

In yet another example, a method for increasing immunity levels in an animal comprises administering to the animal an animal feed composition comprising a feed component comprising one or more of corn, soy, corn or soy derivatives or byproducts, or grains and an added whole cell Euglena biomass, including beta-1,3-glucan comprising at least about 90 percent linear, unbranched beta-1,3-glucan polysaccharide polymers having an average molecular weight of about 1.2 to 580 kilodaltons (kDa) and beta-glucan polymer chains having a polymer length of about 7.0 to 3,400 glucose monomers. The whole cell Euglena biomass comprises at least 30 percent beta-1,3-glucan and includes residual media remaining from a heterotrophic fermentation process that produced the whole cell Euglena biomass. The added whole cell Euglena biomass and residual media are about 0.0001 to 0.0124 percent w/w of the composition.

In still another example, a method for increasing immunity levels in an animal comprises administering to the animal an animal feed composition comprising a feed component comprising one or more of corn, soy, corn or soy derivatives or byproducts, or grains and an added Euglena biomass lysate having an average particle size of about 2.0 to 500 micrometers and comprising cellular components, including beta-1,3-glucan comprising at least about 90 percent linear, unbranched beta-1,3-glucan polysaccharide polymers having an average molecular weight of about 1.2 to 580 kilodaltons (kDa) and beta-glucan polymer chains having a polymer length of about 7.0 to 3,400 glucose monomers. The Euglena lysate comprises at least 30 percent beta-1,3-glucan and includes residual media remaining from a heterotrophic fermentation process that produced a Euglena biomass and the Euglena lysate. The added Euglena lysate and residual media are about 0.0001 to 0.0124 percent w/w of the composition.

### DESCRIPTION OF THE DRAWINGS

Other objects, features and advantages of the present invention will become apparent from the detailed description of the invention which follows, when considered in light of the accompanying drawings in which:

FIG. 1 is a high-level flowchart showing a preferred beta-glucan production process using a repeat fed batch fermentation in accordance with a non-limiting example.

FIG. 2 is another high-level flowchart showing a beta-glucan production process using continuous fermentation in accordance with a non-limiting example.

FIG. 3 is a high-level flowchart showing an example of downstream processing for making purified beta-glucan in accordance with a non-limiting example.

FIG. 4 is a high-level flowchart showing an example of downstream processing for making beta-glucan lysate in accordance with a non-limiting example.

FIG. 5 is a high-level flowchart showing an example of downstream processing for making whole cell Euglena gracilis in accordance with a non-limiting example.

FIG. 6 is a high-level flowchart of a beta-glucan production process using a combination of autotrophic, mixotrophic and heterotrophic in accordance with a non-limiting example.

FIG. 7 is an example of a capsule containing the composition formed from an example Euglena gracilis processing of FIG. 1 in accordance with a non-limiting example.

FIG. 8 is a bar chart showing the results of the pre-clinical trials using whole cell Euglena biomass as an adjuvant in adaptive immunity and showing the relative antibody concentration and percent of Euglena by weight of the animal feed.

FIG. 9A is a bar chart showing the results of the pre-clinical trials using whole cell Euglena biomass to enhance innate immunity and showing the percentage of the phagocytosing blood neutrophils relative to the percentage of Euglena in the animal feed.

FIG. 9B is a bar chart similar to that shown in FIG. 9A, but instead showing the percentage of specific pathogen killing by NK cells.

FIG. 10 is a bar chart showing the results of the pre-clinical trials using whole cell Euglena biomass to increase cellular signaling similar to that shown in FIG. 9B, but showing the interleukin-2.

FIGS. 11A and 11B are charts showing the composition ingredients for the animal feed used in the pre-clinical trials of FIGS. 8 through 10.

### DETAILED DESCRIPTION

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

Beta-glucan from Euglena gracilis is also known by those skilled in the art as: beta-1,3-glucan, beta-1,3-D-glucan, paramylon, algae beta-glucan or Euglena beta-glucan. Below are details of a scaled-up processing method using fermentation of a protist organism known as Euglena gracilis, which usually produces between 50-75% beta-glucan by weight and is stored as intracellular crystalline granules. Some processing may produce up to 83 to 85 percent beta-glucan by weight. Beta-glucan is a glucose polymer and the glucose linkages in the beta-glucan produced by Euglena gracilis are primarily 1,3 and usually greater than 90% and often greater than 94% and can be up to 99%. Other sources of beta-glucan have different ratios of 1,3, 1,4, 1,6, 2,3 and 3,6 linkages, and include branching and different polymer lengths, for example, beta-glucan produced from yeast as compared to beta-glucan produced from Euglena gracilis. These structural differences from other beta-glucan sources are believed to elicit different responses in in vivo animal trials. Alterations to the native beta-1,3-glucan structure with non-limiting functional group substitutions such as acylations, sulfonations, nitrations, phosphorylations or carboxymethylations may beneficially alter the physicochemical properties of the glucan depending on use, for example, to improve solubility, product localization or binding site affinities.

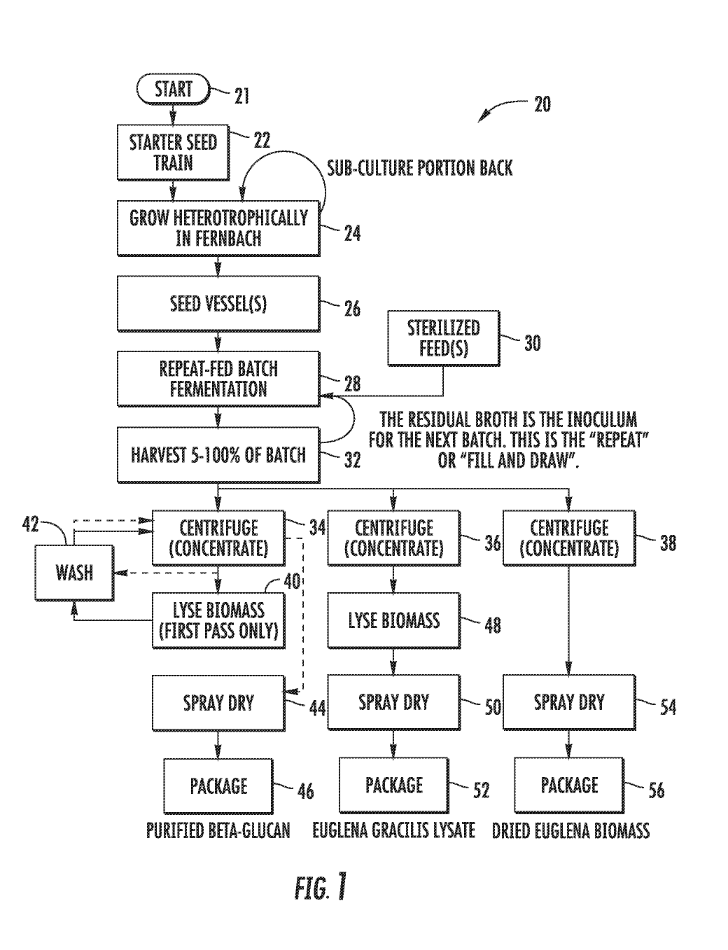

Referring now to FIG. 1, there is illustrated generally at 20 a sequence of processing steps that may be used for producing beta-glucan in accordance with a non-limiting example. The process uses what is referred to as a repeat-fed batch fermentation and produces a composition as purified beta-glucan, a Euglena gracilis lysate or a dried Euglena biomass. It should be understood that the lysate could refer to an aqueous lysate or a dried lysate, but of course, refers to a composition resulting from a lysis as breaking the cells open. Sometimes it may be referred to as a fermentate or an extract. The whole cell biomass may be dried or aqueous.

The process starts (Block 21) with a starter seed train (Block 22) and growing a culture heterotrophically in a Fernbach flask, for example, a standard sized flask known to those skilled in the art (Block 24). A subculture portion is fed back while the other portions are passed into a seed vessel or tank (Block 26) and then to the fermentation tank. At this time, fermentation continues in a repeat-fed batch fermentation process (Block 28) as explained in greater detail below using the sterilized feed (Block 30).

Operationally the fermentation process controls the temperature from 23-32° C., has a pH between 3-5, and a dissolved oxygen content between 10-40% with or without agitation provided by stirring and delivery of air or oxygen. Nutritive sources may include glucose and other sugar or short chain fatty acids as the carbon source, amino acids or ammonia and salts therefrom for nitrogen, and trace metal components and vitamins. At least one of existing and new fermentation growth components may be added to the fermentation batch during fermentation and at least a portion of the fermentation batch may be harvested to produce a biomass.

Approximately 5% to about 95% of the batch is harvested (Block 32) depending on fermentation requirements and operating parameters, and the residual broth is the inoculum for the next batch. This process corresponds to a “repeat” or “draw and fill” process. At this time, the output from the harvesting of about 5% to about 95% of the batch is centrifuged to form a concentrated slurry or wet cake followed by three processing stages starting with a preferred decanter centrifuge shown at respective Blocks 34, 36 and 38 depending on the desired product type in this non-limiting example. It should be understood that the decanter centrifuge separates the solid materials from liquids in a slurry using centrifugal force. Different centrifuge technologies may be used for dewatering instead of a decanter centrifuge, such as a stacked-disk, conical plate, pusher, or peeler centrifuge. They are designed for large scale processing. Gravity decanting and other centrifuge techniques may be used to dewater the biomass in addition to other concentrating techniques such as filtration.

In a first sequence after centrifugation, the biomass is lysed (Block 40) in a first pass only. It is also washed (Block 42) such as during the centrifugation, and after lysing and washing, it is spray dried (Block 44) as an example and packaged (Block 46) as a purified beta-glucan resulting from the wash. The washing process is described below and can vary depending on the cell lysis technique used. To lyse the cells, various mechanical disrupting equipment, chemicals or other specialized lysing operations could be used. In a second possible sequence after centrifugation (Block 36), the biomass is lysed (Block 48) and spray dried (Block 50) to be packaged (Block 52) for a Euglena gracilis lysate. In a third possible sequence after centrifugation (Block 38), it is spray dried (Block 54) and packaged (Block 56) as a dried Euglena gracilis biomass.

As will be explained in greater detail below, the lysate or whole cell material composition may include the fermented material as including those components outside the algae cell that were in the fermentor and included in the composition as formed. The composition may include some media and vitamins, even though many components may have been consumed during the fermentation process. This may include a composition comprising a metal and a beta glucan, in which the metal may be zinc. The composition may include the biomass lysate with proteins and amino acids, lipids, minerals such as the zinc, metabolites, vitamins, and beta-glucan. This combination of cellular fragments and other components may impart further advantageous properties to the final product. Those components outside the biomass that were in the fermentor may become part of the lysate product and composition for advantageous and useful benefits in various and possible dietary, medical, and cosmetic uses.

The starter seed train (Block 22) is now explained with the understanding that a first step in starting a heterotrophic culture is to prepare the media. The seed train may be initiated from a slant, a plate, a frozen culture or other culture storage mechanism. Multiple passages in flasks starting from 50 milliliters up to three liters or more may be used to prepare the culture for the seed vessel(s) and the starter seed train.

When the seed train processing is completed, seed fermentation may occur. In a production scale environment it is typical to have at least one seed vessel with culture passaged into a progressively larger seed vessel, prior to using the largest production fermentation equipment. The purpose of the seed vessel(s) is the same as the seed train: to maximize biomass accumulation. The seed vessel process is typically a batch fermentation process, but includes in one example a sterile feed for some or all media components. It may require aeration and some mixing to prevent biomass settling.

In a production scale environment, the final fermentation tank is usually the largest vessel and may be a limiting step in the overall facility output. The purpose of the production fermentation vessel is to generate the molecule(s) of value. The media used at this stage may include different components and additional changes and alterations to the media may be developed. As compared to the seed train and the overall seed fermentation, this stage of the process will not only accumulate additional biomass, but also will optimize paramylon production. There are several fermentation options for the Euglena gracilis processing. These include: (1) Batch; (2) Fed-Batch; (3) Repeat-Batch; and (4) Continuous Fermentation.

1. In Batch, the media are added prior to inoculation. An additional process to the batch fermentation could be aeration, mixing, temperature control and acid/base components for pH control.

2. In Fed-Batch, additional media may be added either continuously or at a discrete time in the fermentation batch. The feed materials may be a whole media recipe, selected components or new components that are not included in the starting batch media. There can be multiple feeds which can start, stop, and have variable dosing rates at any time during the fermentation. An additional process to the fed-batch fermentation could be aeration, mixing, temperature control and acid/base components for pH control or any combination of the listed.

3. The Repeat-Batch (Repeat-draw) process is a batch fermentation. However, at the end of a batch, a portion of the fermentation may be harvested as compared to a standard batch fermentation where the entire fermentor is harvested. New sterilized media may be added to the residual culture in the fermentor. Repeat batch can allow for higher inoculum amounts than can be delivered by a seed vessel. Additionally the tank turnaround time (downtime) and/or unproductive time may be reduced. A seed vessel is usually necessary to start the repeat-batch series, but may not be required for every batch, which lowers the seed train workload. An additional process to the repeat-batch fermentation could be aeration, mixing, temperature control and acid/base components for pH control or any combination of the listed.

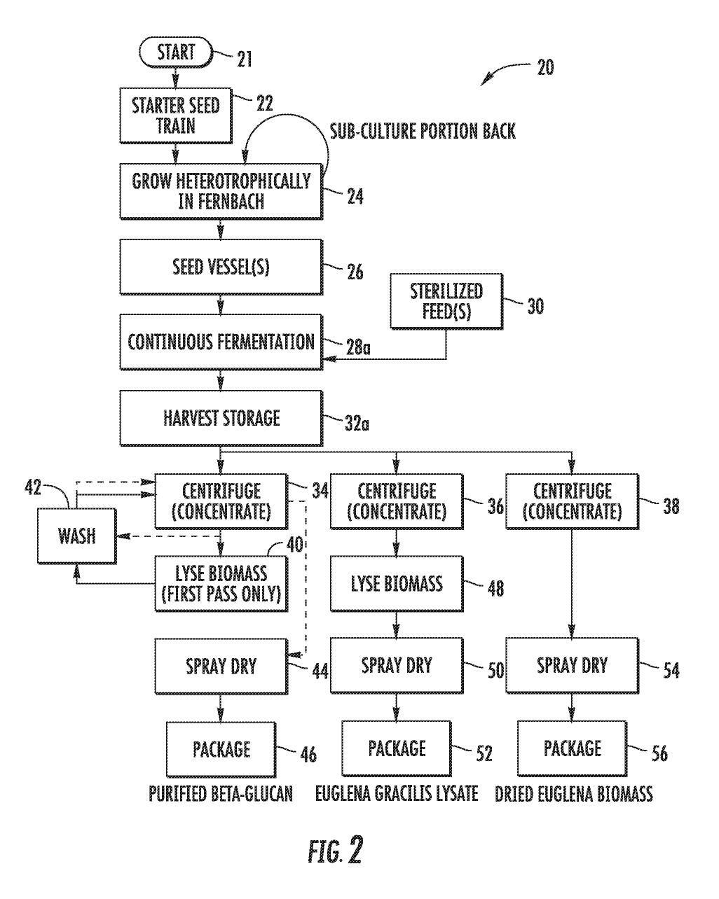

4. In continuous fermentation such as shown in FIG. 2, a stream of sterilized media components or selected components from the original medium or components not outlined in the original medium is fed to the fermentation process, while a continuous purge of the fermentor or fermentation tank is harvested. The fermentation is maintained at a volumetric capacity and a biological balance remains between the inlet nutrients and the outlet harvest flow rates. This fermentation process is never fully harvested, and allows for continual harvest volumes and minimal tank turnaround. An additional process to the continuous fermentation could be aeration, mixing, temperature control and use of acid/base components for pH control or any combination of the listed.

The continuous fermentation process in FIG. 2 is similar to the Repeat-Fed Batch Fermentation except there is a continuous fermentation (Block 28a) instead of the Repeat-Fed Batch Fermentation (Block 28 in FIG. 1). Also, when continuous fermentation is used, there is no harvesting of the 5 to 95% of the batch (Block 32 in FIG. 1) and instead there is a harvest storage to collect the continuous discharge from the fermentor (Block 32a).

There are multiple techniques to produce the dried biomass. A preferred technique would be to mechanically dewater through a decanter centrifuge followed by spray drying. Different centrifuge technologies may be used, such as a stacked-disk, conical plate, pusher, or peeler centrifuge. A spray dry step could produce a flowable powder that can be heated to reduce the microbial bioburden. Additionally, the biomass slurry can be heated prior to spray drying to reduce microbial bioburden in the final material. The biomass can also be ribbon dried, tray dried, freeze dried, drum dried, vacuum ribbon dried, refractance window dried, vacuum drum dried, or dried by other techniques known to those skilled in the art.

The whole lysate of the Euglena biomass is believed to be advantageous for a composition since it may have enhanced bioavailability and other functional benefits. Dried lysate is the dried form of the preferred Euglena gracilis biomass in which the cell membrane, or more specifically the pellicle, has been lysed or disrupted. It should be understood that the lysate may be derived from any species of the genus Euglena. Lysis can occur through mechanical or chemical routes. In a non-limiting example, mechanical cell lysis can occur through homogenization at pressures greater than 350 to 500 barg, including 500 to 1900 barg and a target range of 700 to 1000 barg. In one example, 413 barg has been used to crack the cells open. However, typically and as explained below, the reference of greater than 500 barg is listed as a non-limiting example, understanding the range could be 350 barg and above. An alternative process at an industrial scale would be to mechanically lyse using a bead mill. A non-limiting example of chemical lysis would be lysis from sodium hydroxide (NaOH) or other strong bases such as potassium hydroxide (KOH). In one non-limiting example, to disrupt the cell, a slurry of biomass at a concentration between 3 to 350 grams per liter (g/L), and more preferably, 50 to 175 g/L may be treated with NaOH at a concentration between about 0.05 to about 2 wt % or to a pH greater than 7.0 at a temperature greater than 5° C. An example temperature range may be 50 to 70° C. This combination of temperature and base dosing disrupts the cells without requiring mechanical force. There may be greater bioavailability for the beta-glucan and other metabolites in a lysed form than in a whole-cell form. The resulting dried lysate material may have an average particle size between 2 to 500 micrometers. More specifically, the average particle size may be 5 to 125 micrometers.

A preferred technique to produce dried biomass lysate is to mechanically disrupt a broth at a concentration between 3 to 350 g/L biomass, and more preferably, 50 to 175 g/L biomass. A homogenizer is used at a pressure greater than 500 barg, which has been tested and shown to be effective in homogenization and generating freed beta-glucan granules. An example range of operating a homogenizer may be about 500 to 1,900 barg and more optimally, 750 to 1,000 barg without requiring additional chemicals or additives to the process to lyse the biomass. Alternatively, a bead mill could be used to mechanically lyse the biomass instead of a homogenizer. The resulting lysate material is not washed or separated and it is dried through a spray drying process with the intent to preserve all present solids and non-volatile, soluble components. Some of the cell components that are broken off may become water soluble and there is some loss of material, and there may be some enrichment of beta-glucan. The lysate material can also be ribbon dried, tray dried, freeze dried, drum dried, vacuum ribbon dried, refractance window dried, or vacuum drum dried as alternatives to spray drying. Other drying techniques known to those skilled in the art may be used. This process creates a material with beta-glucan freed from the biomass in addition to value added cellularly produced materials or cellular components with health benefits. There are also different techniques and options for producing purified paramylon therefrom.

### I. Mechanical Disruption

A preferred technique to produce dried purified beta-glucan is to mechanically disrupt a broth at a concentration between 3 to 350 g/L biomass, or more preferably, 50 to 175 g/L biomass. A homogenizer can be used at a pressure greater than 500 barg, which has been tested and shown to be effective in homogenization and generating freed beta-glucan granules. An example range of operating a homogenizer may be about 350 to 500 to 1,900 barg and more optimally, 750 to 1,000 barg without requiring additional chemicals or additives to the process to lyse the biomass. Alternatively, a bead mill could be used to mechanically lyse the biomass instead of a homogenizer. The lysed material may be washed with water to remove cellular components. Additional washing may be performed using a base, acid, water or a combination therein. A base, for example, sodium hydroxide (NaOH) may be added to the lysed slurry at a 0.05 to 2.0 wt % concentration or to a pH greater than 7.0. It is possible to use other bases such as potassium hydroxide (KOH) and ammonium hydroxide (NH₄OH) as non-limiting examples. Additional washes with water or 0.05 to 2.0 wt % caustic (NaOH) solutions can be completed. An acid wash is possible. For example, sulfuric acid may be added between 0.05 to 1.0 wt % or to a solution pH between 2.0 to 10.0 and preferably 3.0 to 5.0. A final water wash may be made subsequent to the acid wash. Other possible acids may include hydrochloric acid (HCl), phosphoric acid (H₃PO₄), and citric acid (C₆H₈O₇) as non-limiting examples. Washing can also be accomplished by using ethanol and with any combination of the treatments above. The beta-glucan slurry or cake should be dewatered between each washing step. Dewatering can occur with centrifugation or decanting after gravity settling. The resulting washed beta-glucan slurry or cake can be spray dried. Alternatively, the material can be dried by a ribbon dryer, vacuum ribbon dryer, drum dryer, tray dryer, freeze dryer, refractance window dryer, vacuum dryer, or dried by other techniques known to those skilled in the art.

### II. Surfactant

A second technique to produce purified beta-glucan involves the treatment of a broth at a concentration between 3 to 350 g/L biomass, and more preferably, 50 to 175 g/L biomass with a surfactant such as sodium dodecyl sulfate (SDS) in concentrations of 0.2 to 2.0 wt %. This solution is heated to between about 50° C. to about 120° C. with a temperature target of about 100° C. for at least 30 minutes. This heated step in the presence of SDS disrupts the cell membrane and frees the intra-cellular paramylon crystal granules.

The slurry may be allowed to gravity decant for about 4 to 24 hours, while the crystal granules settle to the bottom of a reactor/decanter tank. The concentrated bottoms are pumped away for additional processing and the remaining liquid is sent to waste. Alternatively, the material can be centrifuged to remove the bulk liquid in lieu of a gravity decant. Different centrifuge technologies may be used, such as a stacked disk, conical plate, pusher, or peeler centrifuge. A food-grade siloxane-based antifoam, such as Tramfloc 1174® or Xiameter 1527®, added in greater than 20 ppm, more specifically 200 to 400 ppm may be used to reduce foaming caused by SDS. The anti-foam can be added before or after the SDS/heat treatment if it is used. The resulting material may be washed with water. The resulting crystal slurry or cake can be spray dried. Alternatively, the material can be dried by a ribbon dryer, vacuum ribbon dryer, drum dryer, tray dryer, freeze dryer, refractance window dryer, vacuum dryer, or dried by other techniques known to those skilled in the art.

### III. Natural Oil Surfactant

A third technique to generate purified beta-glucan involves the treatment of a broth at a concentration between 3 to 350 g/L biomass, and more preferably, 50 to 175 g/L biomass with a surfactant produced from natural oils such as sodium cocoyl glycinate or sodium N-cocoyl-L-alaninate (Amilite® ACS12) derived from the fatty acids in coconut oil in an amount of about 0.2 to about 5.0 wt %. This solution is heated to between about 50° C. to about 120° C. with a current target of about 100° C. for at least 30 minutes. This heat step in the presence of sodium N-cocoyl-L-alaninate or sodium cocoyl glycinate disrupts the cell membrane and frees the intra-cellular paramylon crystal granules. The time, temperature, and concentration parameters may be refined depending on the exact surfactant used.

The slurry is allowed to gravity decant for about 4 to 24 hours while the crystal granules settle to the bottom of a reactor/decanter tank. The concentrated bottoms may be pumped for additional processing while the remaining liquid is sent to waste. Alternatively, the material may be processed through a centrifuge to remove the bulk liquid in lieu of a gravity decant. Different centrifuge technologies may be used, such as stacked-disk, conical plate, pusher, or peeler centrifuging. An anti-foam may be added. An example anti-foam material is a food-grade siloxane-based antifoam, for example, Tramfloc 1174® or Xiameter 1527®. The anti-foam may be used to reduce foaming caused by the surfactant. The anti-foam may be added before or after the surfactant/heat treatment if it is applied. An example dosing range includes an amount greater than 20 ppm, more specifically 200 to 400 ppm. The resulting material may be washed with water. The resulting crystal slurry or cake can be spray dried. Alternatively, the material can be dried by a ribbon dryer, vacuum ribbon dryer, drum dryer, tray dryer, freeze dryer, refractance window dryer, vacuum dryer, or dried by other techniques known to those skilled in the art.

Amino acid-based surfactants derived from coconut oil fatty acids are anionic and demonstrate a lower potential for outer layer skin damage, while also exhibiting equal or greater cleansing ability. These attributes are described in the article by Regan et al. entitled, “A Novel Glycinate-Based Body Wash,” Journal of Clinical and Aesthetic Dermatology, June 2013; Vol. 6, No. 6, pp. 23-30, the disclosure which is hereby incorporated by reference. Sodium cocoyl glycinate (SCG) is composed of N-terminally linked glycine with a spectrum of fatty acids in natural coconut oil containing carbon lengths and percentages of 10, 12, 16, 18:1 and 18:2 and 6, 47, 18, 9, 6 and 2 respectively such as described in the report from National Industrial Chemicals Notification and Assessment Scheme, Sodium Cocoyl Glycinate, EX/130 (LTD/1306), August 2010, the disclosure which is hereby incorporated by reference. Both sodium N-cocoyl-glycinate and sodium N-cocoyl-L-alaninate are examples of coconut oil derived surfactants. It is possible to use surfactants derived from palm oil, palm kernel oil, and pilu oil, which are similar to coconut oil based on the ratios and distribution of the fatty acids sized from C8 to C18. Coconut oil contains a large amount of lauric acid (C12) but also a significant amount of caprylic (C8), decanoic (C10), myristic (C14), palmitic (C16), and oleic acids (C18). Palm oil, palm kernel oil, and pilu oil have similar fatty acid profiles as coconut oil which means surfactants derived from these oils could be equally effective than surfactants derived from the fatty acids in coconut oil. These may also be suitable alternatives to SDS. The ranges and content of these fatty acids as naturally derived surfactants may vary.

### IV. pH Mediated Lysis

A fourth technique to produce purified beta-glucan is to chemically disrupt the biomass using a base. A non-limiting example would be lysis from sodium hydroxide (NaOH) or other bases such as potassium hydroxide (KOH). In one non-limiting example, to disrupt the cell, a slurry of biomass at a concentration between 3 to 350 grams per liter (g/L), and more preferably, 50 to 175 g/L may be treated with NaOH at a concentration between about 0.05 to about 2 wt % or to a pH greater than 7.0 at a temperature greater than 5° C. A non-limiting example temperature range may be 45 to 70° C. and pH range may be 9.0 to 12.5. This combination of temperature and base dosing disrupts the cells without requiring mechanical force. A first treatment with the base should lyse the cells. If too little base is applied or the temperature is too low, the cells may not be disrupted, and if too much base is applied and/or the temperature is too high, most components and the beta-glucan may go into solution. Washing with water may be performed. Additional washing may be performed using a base, an acid, or water in sequence or any combination, such as acid, a base, and then water.

Additional washes with water or 0.05 to 1.0 wt % sodium hydroxide (NaOH) solutions or to a pH greater than 7.0 can be completed. Potassium hydroxide (KOH) will also work. Other possible bases include ammonium hydroxide (NH₄OH) as a non-limiting example. An acid wash may be completed. For example, sulfuric acid may be added between 0.05 to 1.0 wt % or to a solution pH between 2.0 to 10.0 and preferably 3.0 to 5.0 can be completed and a final wash with water may be made subsequent to the acid wash. Other possible acids may include nitric acid (HNO₃), hydrochloric acid (HCl), phosphoric acid (H₃PO₄), and citric acid (C₆H₈O₇) as non-limiting examples. Washing can also be accomplished by using ethanol and with any combination of the treatments above. The beta-glucan slurry or cake should be dewatered between each washing step. Dewatering can occur with centrifugation or gravity decanting. Different centrifuge technologies may be used, such as a stacked disk, conical plate, pusher, or peeler centrifuge. The resulting washed beta-glucan slurry or cake can be spray dried. Alternatively, the material can be dried by a ribbon dryer, vacuum ribbon dryer, drum dryer, tray dryer, freeze dryer, refractance window dryer, vacuum dryer, or dried by other techniques known to those skilled in the art.

### V. Enzymatic Treatment

A fifth technique to produce purified beta-glucan focuses on enzymatic treatment. Cell lysis may occur through mechanical disruption or other treatments as described above and the biomass can be at a concentration between 3 to 350 g/L, and more preferably, 50 to 175 g/L. Cell lysis prior to treatment may also not be required. The pH and temperature of the slurry can be adjusted with an acid or base and energy to meet the conditions required for optimal enzymatic treatment. A non-specific protease can be used to degrade proteins from the cells. A non-limiting example could be Alcalase® 2.4L FG from Novozymes. The resulting enzymatically treated slurry can be washed with an acid, base, ethanol, or water, or any combination therein, in order to remove the enzymatically treated components and then dewatered. Dewatering can occur with centrifugation or gravity decanting. Different centrifuge technologies may be used, such as a stacked disk, conical plate, pusher, or peeler centrifuge. The resulting beta-glucan slurry or cake can be spray dried. Alternatively, the material can be dried by a ribbon dryer, vacuum ribbon dryer, drum dryer, tray dryer, freeze dryer, refractance window dryer, vacuum dryer, or dried by other techniques known to those skilled in the art. Other enzymes such as a lipase may be used in addition to the protease. Another example is a lysozyme used alone or in combination. Additionally, an enzyme deactivation step may be required. The amount of post enzyme treatment washing may be determined during processing but could follow the processes outlined above.

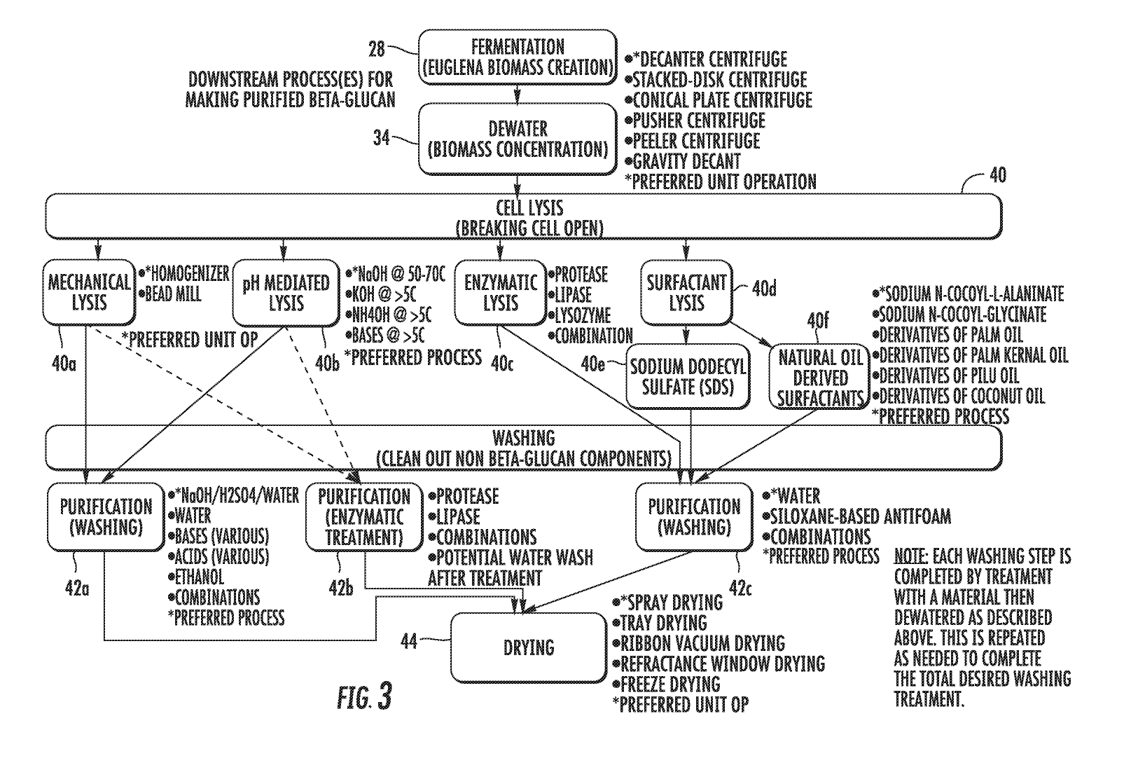

FIG. 3 is a flowchart showing downstream processes for making the purified beta-glucan. Reference numerals corresponding to those shown in FIG. 1 are used with reference to the general description of flow components as in FIG. 1. The fermentation process creates the Euglena biomass (Block 28) that is dewatered to concentrate the biomass (Block 34). Dewatering could include processing by the preferred decanter centrifuge or the other centrifuge techniques including stacked-disc, conical plate, pusher and peeler centrifuging. It is also possible to use gravity decantation. As a one pass process of FIG. 1, the cell lysis process disrupts the cellular pellicle and can be accomplished using a mechanical lysis (Block 40a), including the preferred homogenizer or bead mill as described above. A pH mediated lysis (Block 40b) may include sodium hydroxide (NaOH) as a preferred base at approximately 50 to 70° C. with other possibilities and further processing including KOH at greater than 5° C., NH₄OH at greater than 5° C. and other bases at greater than 5° C. Another example may include enzymatic lysis (Block 40c) and may include protease, lipase, lysozyme or a combination of those processes. The protease is an enzyme that catalyzes proteolysis with the use of water to hydrolyze protein and peptide bonds while the lipase enzyme catalyzes the hydrolysis of lipids. A lysozyme enzyme typically operates as a glycoside hydrolase.

Another example of the cell lysis process includes using a surfactant lysis (Block 40d) such as using sodium dodecyl sulfate (SDS) (Block 40e) or a natural oil derived surfactant (Block 40f), including sodium N-cocoyl-L-alaninate or sodium N-cocoyl-glycinate. Other possible natural oil derived surfactants include derivatives of palm oil, derivatives of palm kernel oil, derivatives of pilu oil, and derivatives of coconut oil. The washing step (Block 42) cleans out the non-beta-glucan components and may include a purification by washing (Block 42a). This may include adding a base and acid with water and any combinations for the preferred process, including sodium hydroxide (NaOH) followed by sulfuric acid (H₂SO₄), and water. The purification may occur by enzymatic treatment (Block 42b) that includes the protease, lipase, or combinations with the potential water wash at the treatment. Purification may also occur by washing (Block 42c) with water and a siloxane-based anti-foam or a combination. The final step of drying (Block 44) may include a preferred spray drying or tray drying, vacuum ribbon drying, refractance window drying, freeze drying, ribbon drying, drum drying, or vacuum drying as alternatives, as well as other techniques known to those skilled in the art.

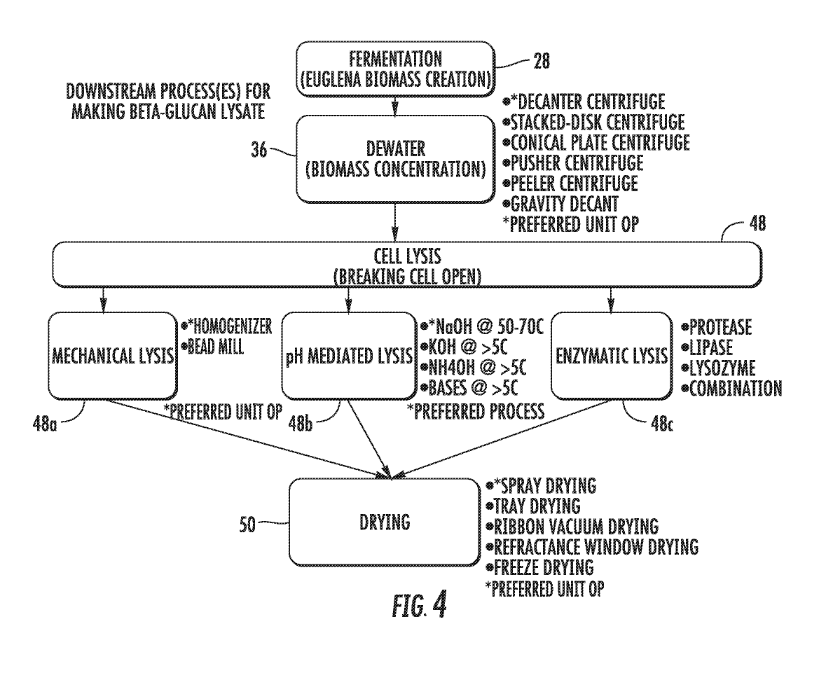

FIG. 4 is a flowchart showing downstream processes for making the beta-glucan lysate. Reference numerals corresponding to those shown in FIG. 1 are used with reference to the general description of flow components as in FIG. 1. The fermentation process creates the Euglena biomass (Block 28) that is dewatered to concentrate the biomass (Block 36). Dewatering could include processing by the preferred decanter centrifuge or the other centrifuge techniques including stacked-disk, conical plate, pusher, and peeler centrifuging. It is also possible to use gravity decantation. The cell lysis process disrupts the cellular pellicle (Block 48) and can be accomplished using a mechanical lysis (Block 48a), including the preferred homogenizer or bead mill as described above. A pH mediated lysis (Block 48b) may include sodium hydroxide (NaOH) as a preferred base at approximately 50 to 70° C. with other possibilities and further processing, including KOH at greater than 5° C., NH₄OH at greater than 5° C., and other bases at greater than 5° C. Another example may include enzymatic lysis (Block 48c) and may include protease, lipase, lysozyme, or a combination of these processes. Drying occurs (Block 50) with a preferred spray drying and may include tray drying, ribbon vacuum drying, refractance window drying, and freeze drying.

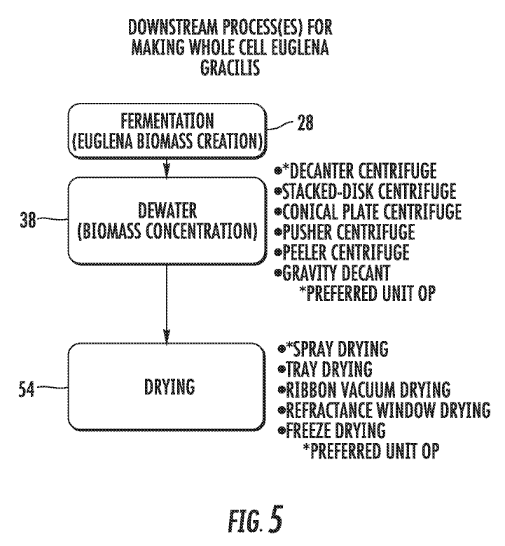

FIG. 5 is a flowchart showing downstream processes for making the whole cell Euglena gracilis. Again, reference numerals corresponding to those shown in FIG. 1 are used with reference to the general description of flow components as in FIG. 1. The fermentation process creates the Euglena biomass (Block 38) that is dewatered to concentrate the biomass (Block 38). Again, the decanter centrifuge is the preferred operation and other processes as described relative to FIG. 4 may also be used. Drying occurs (Block 54) with spray drying as preferred and with other drying techniques that may be applicable as described with reference to FIG. 4.

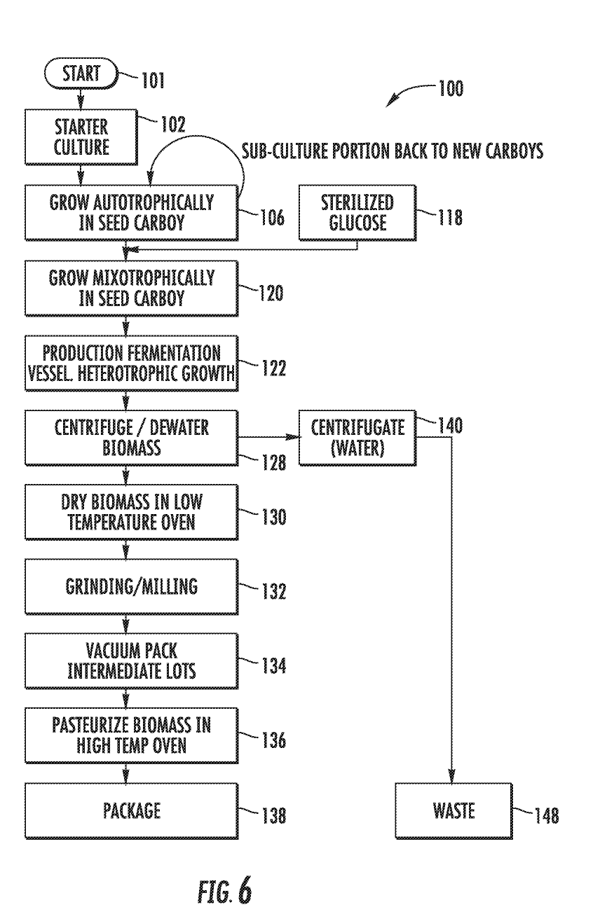

Another example of a beta-glucan production process is shown in FIG. 6 at 100 and shows a method for producing beta-1,3-glucan using a combination of autotrophic, mixotrophic, and heterotrophic growth techniques. As a high level description, the beta-1,3-glucan is produced by culturing Euglena gracilis. The starting culture for the process may be initiated from starter slants or other stored culture source. It is then grown autotrophically. This is followed by converting the batch to mixotrophic growth by adding glucose. The mixotrophic material is then used to inoculate a heterotrophically operated Euglena gracilis fermentation.

As explained further in the flowchart of FIG. 6, the process (Block 100) starts (Block 101) and a starter slant is prepared (Block 102). The Euglena gracilis seed culture is grown autotrophically in a seed carboy (Block 106) with the subculture portion fed back to new carboys.

After the Euglena gracilis seed culture is grown autotrophically, it is fed sterilized glucose (Block 118), which converts it into a mixotrophic seed carboy (Block 120). The autotrophically grown Euglena gracilis seed culture is now grown mixotrophically for about 7 to about 30 days and then used to inoculate a fermentation tank where heterotrophic fermentation occurs for about 4 to about 7 days (Block 122). This process of heterotrophic fermentation occurs for about 4 to about 7 days to produce beta-glucan rich Euglena gracilis. A Euglena gracilis biomass is removed and dewatered by a centrifugation (Block 128) followed by drying (Block 130) in an oven. The biomass cake is dried at about 80° C. to 120° C. Once dry, the material may be ground and milled (Block 132) followed by screening and vacuum packing (Block 134) followed by pasteurization (Block 136). The pasteurization temperature range may vary and in one example may be about 160° C. and run for no less than 2 hours. After pasteurization, the product may be packed for human or animal use (Block 138). Also, the centrifugate as water (Block 140) is processed as waste (Block 142).

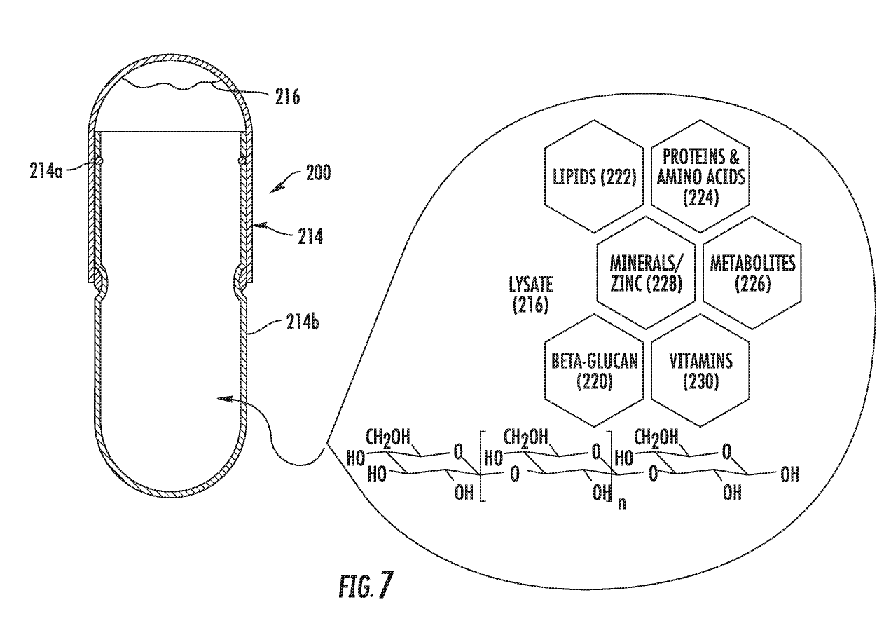

Referring now to FIG. 7, a lysate composition delivery system 200 includes a capsule 214 containing the final product as the lysate 216 produced from the process such as described in FIG. 1. The capsule 200 may be formed from conventional upper and lower capsule sections 214a and 214b. However, other delivery mechanisms such as tablets, powders, lotions, gels, liquid solutions and liquid suspensions are also possible.

As shown by the enlarged section of final product as a lysate 216 taken from the material within the capsule, the capsule material 216 contains not only a linear, unbranched beta-glucan 220, but also other material from the fermentor that creates an enhanced composition. These components may include lipids 222, proteins and amino acids 224, metabolites 226, minerals such as zinc 228 and vitamins 230, and other value added, cellularly produced components and cellular materials. This composition therefore includes in one example a Euglena lysate additionally including cellular components and residual media remaining from the fermentation batch that produced the Euglena lysate. The composition also includes various additive metal components such as zinc. An example range for metal components, including zinc, are 0.1 to 10 wt %.

In an example, the composition is delivered in a single dosage capsule. Some of the beta-glucan components may include one or more beta-glucan polymer chains and vary in molecular weight from as low as 1.2 kDa to as high as 580 kDa and have a polymer length ranging from as low as 7 to as high as 3,400 glucose monomers as one or more polymer chains. The beta-glucan polymers can exist individually or in higher order entities such as triple helices and other intermolecularly bonded structures dependent upon fermentation or processing conditions. An example mean particle size range could be 2.0 to 500 micrometers (microns) for the lysate produced by the processes as described. More specifically, the average particle size may be 5 to 125 micrometers. This range may vary depending on processing parameters and drying technology used. Other components that may be included within the lysate composition include carotenoids such as alpha- and beta-carotene, astaxanthin, lutein, and zeaxanthin. Amino acids may be included such as alanine, arginine, aspartic acid, cysteine, cystine, glutamic acid, glycine, histidine, isoleucine, leucine, lysine, methionine, phenylalanine, proline, serine, threonine, tryptophan, tyrosine and valine. Other lipids, vitamins and minerals include arachidonic acid, biotin, calcium, copper, docosahexaenoic acid, eicosapentaenoic acid, fats, folic acid, iron, linoleic acid, linolenic acid, magnesium, manganese, niacin, oleic acid, palmitoleic acid, pantothenic acid, phosphorus, potassium, protein, sodium, vitamin B1, B2, B6, B12, C, D, E, K1, zinc or salts therefrom, as well as leftover components from the Euglena algae, including other cellular components not listed above and added media obtained from fermentation.

The ranges of supplementation may vary. For example, as a dietary supplement composition for human consumption, the composition can range from 50 to 12,000 mg per kilogram of food or from about 50 mg to 2,000 mg as a capsule dosage. These amounts can vary depending on the end uses and may vary even more when used for other uses. In certain examples, this may include animal uses. For example, animal ranges could be from 10 mg/kg to 5,000 mg/kg as a blend in feed. Other ranges could be 50 to 6,000 mg.

There now follows a listing of ranges for the different components of the lysate. These ranges are for the lysate as produced and do not include other components added to the lysate, for example, zinc. These non-limiting examples are approximate weight percentages for components or compounds identified in the Euglena lysate.

TABLE 1
Vitamins and Minerals
Vitamins and Minerals (<2%)

| | Percentage of |
|---|---|
| Compound | Lysate (w/w) |
| biotin | <0.1 |
| calcium | <0.1 |
| copper | <0.1 |
| folic acid | <0.1 |
| iron | <0.1 |
| magnesium | <0.1 |
| manganese | <0.1 |
| niacin | <0.1 |
| phosphorus | <0.1 |
| potassium | <0.1 |
| sodium | <0.1 |
| zinc | <0.1 |
| vitamin B2 | <0.1 |
| vitamin B6 | <0.1 |
| vitamin B12 | <0.1 |
| vitamin C | <0.1 |
| vitamin D | <0.1 |
| vitamin E | <0.1 |
| vitamin K1 | <0.1 |

TABLE 2
Protein and Amino Acids
Protein and Amino Acids (10-20%)

| | Percentage of |
|---|---|
| Compound | Lysate (w/w) |
| peptides and protein | 8-18 |
| alanine | <1 |
| arginine | <0.5 |
| aspartic acid | <1 |
| cysteine | <0.1 |
| cysteine | <0.1 |
| glutamic acid | <1 |
| glycine | <1 |
| histidine | <0.5 |
| isoleucine | <0.1 |
| leucine | <0.5 |
| lysine | <0.5 |
| methionine | <0.1 |
| phenylalanine | <0.1 |
| proline | <0.1 |
| serine | <1 |
| threonine | <0.5 |
| tryptophan | <0.5 |
| tyrosine | <0.1 |
| valine | <0.5 |

TABLE 3
Fats
Fats (5-20%)

| Compound | Percentage of Lysate w/w) |
|---|---|
| linoleic acid | <1 |
| linolenic acid | <1 |
| oleic acid | <1 |
| palmitoleic acid | <1 |
| pantothenic acid | <1 |
| arachidonic acid | <1 |
| docosahexaenoic acid | <2 |
| eicosapentaenoic acid | <2 |
| other fats | 2-10 |

TABLE 4
Other Constituents
Other Constituents (40-90%)

| Compound | Percentage of Lysate (w/w) |
|---|---|
| paramylon | 30-80 |
| alpha carotene | <1 |
| beta carotene | <1 |
| lutein | <1 |
| astaxanthin | <1 |
| zeaxanthin | <1 |
| water | 0.5-10 |

The desired response from glucan supplementation can vary. For example, soluble and particulate beta-glucans have elicited biological effects beyond immune modulation. There is evidentiary support for antimicrobial, antiviral, antitumoral, antifibrotic, antidiabetic and anti-inflammatory responses as well as evoking microbiome sustenance, in the form of a prebiotic, hepatoprotective, hypoglycemic, cholesterol lowering, wound healing, bone marrow trauma and radiation and rhinitis alleviating effects. The bioactivities mentioned are triggered by glucans and may then have potential applications in treatments of viral and bacterial infection, cancer, cardiovascular disease, liver disease, blood disorders, diabetes, hypoglycemia, trauma, skin aging, aberrant myelopoiesis, arthritis, microbiome deficiencies, ulcer disease and radiation exposure. Additionally outside the scope of human health, beta glucan has potential applications in animal husbandry. Beta glucans can potentially improve growth performance by allowing the livestock to grow at optimal rates through immune modulation to combat growth rate deterrents such as disease and environmental challenges common to the trade. In addition to the potentially synonymous benefits intended for humans previously mentioned, beta glucans could specifically provide preventative measures in contracting significant animal diseases in non-limiting examples such as Porcine Respiratory and Reproductive Syndrome (PRRS), Porcine Epidemic Diarrhea virus (PEDv), Newcastle disease and avian influenza. Additionally beta glucans can have absorptive effects for mycotoxins produced by fungal infection. This indicates potential for preventing mycotoxin production by having fungicidal activity initially or clearing mycotoxin accumulations in animals from mycotoxin contaminated feed ingestion.

Synergistic effects may be observed with addition of beta glucan derived products with other natural foods and remedies including Echinacea, aloe, golden seal, ginseng, garlic, bell peppers, ginger, tumeric, gingko biloba, cat's claw, ganoderma or astragalus. It may be mixed further with vitamin C and possibly humic and fulvic acids. It is also possible to mix glucan with resveratrol or other polyphenols and work for treating heart disease and possibly cancer. Other foods and remedies that may be added include broccoli, spinach, and other leafy vegetables, yogurt, almonds, honey, green tea, papaya, kiwi, poultry, shrimp, sunflower, vitamin D, mushrooms, pumpkin, cinnamon, parsnips, grapes, sweet potatoes, milk, orange juice, rice, carotenoids, figs, glutamine, arginine, an omega-3 fatty acid, vitamin A, vitamin E, selenium, zinc, a probiotic, corn, soy or corn or soy derivatives, including dried distiller grains.

Throughout this description, the term beta-glucan alone or beta-1,3-glucan will typically refer to the beta-glucan produced as a lysate or purified beta-glucan described above relative to the drawing figures unless indicated or described otherwise. For purposes of explanation, the term linear or unbranched beta-glucan may be used for this beta-1,3-glucan as described, since it includes a majority of the described, linear, unbranched beta-1,3-glucan. The term Euglena derived beta-glucan also refers to that produced from the processes described above and shown in the drawing figures. Dried whole cell Euglena biomass refers to the whole cell Euglena produced from the heterotrophic fermentation process such as described in FIG. 5.

An example lysate composition that is formed such as in the process described relative to FIG. 4 is listed below in Table 5 showing the component list for the lysate.

TABLE 5
Component List for Lysate

| | Amount | | Amount |
|---|---|---|---|
| Component | (per serving) | Units | (g/100 g) |
| Proximates: | | | |
| Protein | 49.6 | mg | 17 |
| Fat: | 31.3 | mg | 10 |
| Saturated fatty acids | 25.7 | mg | 9 |
| Mono-unsaturated fatty acids | 2.5 | mg | 1 |
| Poly-unsaturated fatty acids | 3.0 | mg | 1 |
| β-1,3-glucan | 200.0 | mg | 67 |
| Moisture | 4.2 | mg | 1 |
| Digestible carbohydrates | — | mg | — |
| Dietary fiber | 223.6 | mg | 75 |
| Cholesterol | — | mg | — |
| Calories | 1.3 | | 438/100 g |
| Digestible Calories | 0.5 | | 164/100 g |
| Minerals: | | | |
| Sulfur | 0.4 | mg | 0.149 |
| Phosphorus | 2.1 | mg | 0.687 |
| Potassium | 0.1 | mg | 0.030 |
| Magnesium | 0.1 | mg | 0.020 |
| Calcium | 0.3 | mg | 0.109 |
| Sodium | 0.1 | mg | 0.020 |
| Iron | 64.5 | mcg | 0.0215 |
| Manganese | 1.8 | mcg | 0.0006 |
| Copper | 2.0 | mcg | 0.0007 |
| Zinc | 0.9 | mcg | 0.0003 |
| Vitamins: | | | |
| Vitamin A (Beta-Carotene) | 1.47 | IU | 490 |
| Vitamin B1 (Thiamine HCl) | 0.40 | mcg | 0.000134 |
| Vitamin B2 (Riboflavin) | 0.04 | mcg | 0.000012 |
| Vitamin B3 (Niacin) | 0.16 | mcg | 0.000052 |
| Vitamin B5 (Pantothenic acid) | 0.14 | mcg | 0.000045 |
| Vitamin B6 (Pyridoxine) | 0.04 | mcg | 0.000012 |
| Vitamin B7 (Biotin) | 0.01 | mcg | 0.000003 |
| Vitamin B9 (Folic acid) | 0.04 | mcg | 0.000012 |
| Vitamin B12 (Cobalamins) | — | mcg | — |
| Vitamin C (Stay C-35) | 41 | mcg | 0.0136 |
| Vitamin E (alpha-tocopherol) | 0.01 | IU | 3 |
| Choline chloride | 366 | mcg | 0.122 |
| Glucosamine (HCl total) | 70 | mcg | 0.023 |

As noted before, the composition may include a dried Euglena biomass lysate having an average particle size of about 2.0 to 500 micrometers (microns), and in a preferred range of about 2.0 to 125.0 micrometers. One embodiment has a range of about 2 to about 40 or 50 micrometers with an average of about 15 to 25 micrometers. It could range at a lower size of about 0.25 to about 4 to 10 micrometers, but in one example, about 5.0 micrometers and ranging from about 5.0 to about 10.0, 10 to 20, or 10 to 50 micrometers with variations of 10% to 20% from these values.

Cellular components formed from lysing the heterotrophically grown Euglena biomass include beta-1,3-glucan that are primarily the linear, unbranched beta-1,3-glucan polysaccharide polymers having a molecular weight of about 1.2 to 580 kilodaltons (kDa) and beta-glucan polymer chains having a polymer length of about 7.0 to 3,400 glucose monomers. In an embodiment, the polysaccharide polymers can range from about 100 to 550 kDa, and in another embodiment, closer to 200 to 500 kDa, and about 150 to about 350 kDa, and a preferred average of about 140 kDa to about 150 kDa in an example.

There may be some branched beta-1,6 side chains forming the branched beta-glucan structure, but the amount will be limited. For example, the linear, unbranched beta-1,3-glucan will usually be at least about 90% of the total beta-glucan with possibly smaller amounts of branched beta-glucan. Typically, a producer of the composition will guarantee at least about 90% of the linear, unbranched beta-1,3-glucan, but in some cases, it could be smaller at about 75%, 80% or 85%. In other examples, the linear, unbranched beta-1,3-glucan is up to about 91%, 92%, 93%, 94%, or 95%, and possibly exceed that and extend as high as 99%. It could be mixed with other branched beta-1,3-glucan such as from yeast if more branched varieties are desired. Paramylon from Euglena gracilis is mostly formed from the linear, unbranched beta-1,3-glucan.

The polymer length for the glucose monomers can vary, of course, depending on the molecular weight. In the linear, unbranched beta-1,3-glucan chains, the glycosidic linkage pattern governs the structure and there may be rotations around the bonds of the glycosidic linkages and forming in one example predominantly a triple-helix backbone and having different types of hydrogen bonding such as the intermolecular hydrogen bonding through the different chains in the same x/y plane and the intramolecular hydrogen bonding adjacent oxygen atoms in the same chain and intermolecular hydrogen bonding between different chains in a different x/y plane. The triple-helix structure can have its hydrogen bonds destabilized to change its conformation and it can be in a native state, denatured, or denatured and renatured. This can affect water solubility so that it can be injected as compared to a particulate beta-1,3-glucan that is used more typically for oral administration such as a lysate and may have much of the triple-helix structure. Some studies indicate that the beta-1,3-glucans produced by Euglena have a molecular weight of about 200 to 500 kDa. This can be broken into smaller units.

It is possible to take the branched variety of beta-glucan and irradiate it for a predetermined time and produce a broken, smaller beta-glucan that may have units that are more linear and have a smaller molecular weight. This irradiated, branched beta-glucan could be added to that linear beta-glucan derived from Euglena heterotrophically from the processes as described above. It is possible to dissolve a highly branched beta-glucan from a yeast or similar source in a solvent and extract the beta-glucan, and irradiate it and break its bonds and degrade the molecules to a low kDa value, which could range from as low as 10 or 20 and possibly 30 to about 60, 70 or 80 kDa. There may be some fragmented beta-1,3-glucans and other branched and beta-1,4-glucans and beta-1,6-glucans and this type of mixed product may not be as preferred as an additive to the linear beta-glucan.

The composition includes in an example the residual media remaining from the heterotrophic fermentation process that produced that Euglena biomass and the Euglena lysate. In yet another example, it may include added vitamin C, or added resveratrol or both. It is also possible to add dried whole cell Euglena biomass to the lysate. Humic acid may be added. As a dietary supplement composition, it may be formulated into a single dosage capsule or spread over several capsules for daily dosage and the dried Euglena lysate, whole cell Euglena biomass, residual media, added vitamin C and added resveratrol with or without humic acid may be from about 50 mg to about 2,000 mg per capsule dosage as noted above, but can vary from 50 mg to 1,500 mg, or 50 mg to 1,000 mg, or start from 100 mg and extend to 500 or 600 mg or higher.

The weight ratios of the beta-glucan to vitamin C may be as high as 40:1 to 1:60, but a preferred ratio is 1:10 to 1:1. That preferred ratio has been advantageous in function with the benefits of both components having what appears to be better efficacy without having excess algae taste and enhancing the effect of each other. It is also possible to blend the purified beta-glucan such as produced from the process shown in FIG. 3 with the lysate such as produced from the process shown in FIG. 4 to boost the beta-glucan content up to greater than 85% in one example. The lysate produced by the described process has an average beta-glucan content of about 60%, but can be higher depending on production such as 67%, and thus, range from about 60% to 70%. However, the beta-glucan content may be as high as about 85% based on just the processing or by adding the purified beta-glucan to the lysate. Often it is desirable in some cases to add purified beta-glucan to raise the beta-glucan up to 75%, 80%, and desirably up to about 85% or higher, such as found with the percentage of branched beta-glucan in some yeast-based products. It has been determined that the beta-1,3-glucan of about 60% content based on dried Euglena biomass lysate and having the linear, unbranched beta-1,3-glucan polysaccharide polymers is as effective as an 85% branched beta-glucan content derived from yeast. Thus, boosting the linear, unbranched beta-1,3-glucan content up to or greater than 85% by adding the purified beta-glucan to the lysate is advantageous. In some cases, the beta-glucan content may be about 30% and this could be accomplished if the Euglena is grown autotrophically, which on an average, makes about 35% glucan. Greater percentages can then be obtained such as by adding heterotrophically grown amounts, including the purified form.

Residual media may remain from the heterotrophic fermentation process that produced the Euglena biomass and the Euglena lysate in the final product as the lysate and also in the dried whole cell. The residual media may be up to 10% of the initial fermentation concentration, but more likely, it will be in the range of 1% to 4% of the initial fermentation concentrations with possible 10% to 20% variations in this example. As to the lysate, it could vary in range for the immune function from about 50 mg to 2,000 mg of lysate for daily administration, typically used orally and ideally about 250 mg per day of the lysate for immune function, which could vary from this value about 10% to 20%. There could be an exception with ranges for cardiovascular uses that would be higher. For example, it is possible to range up to about 3,000 to 5,000 mg per day of the lysate for cardiovascular uses in one example. This 3,000 to 5,000 mg per day of lysate for cardiovascular usages could be distributed over a number of capsules, and the values may range 10% to 20%. The use of the dried whole cell Euglena biomass could have similar ranges.

As noted before, it is possible to process the lysate to have about 85% beta-glucan, and in other examples, add purified beta-glucan as described above to the lysate, allowing in some examples the Euglena biomass lysate, and in one example, a dried lysate to be at least 85% beta-1,3-glucan with the added purified linear, unbranched beta-1,3-glucan. For example, 50 mg to 250 mg of the purified beta-glucan may be added to about 250 mg of lysate in an example, with variations of about 10% to 20% for each. In another example, even fewer amounts of the purified beta-glucan can be added. In one example, based on 250 mg of lysate, from 10% to 100% of that weight of purified beta-glucan could be added corresponding to as low as 25 mg of added purified beta-glucan, with values varying depending on the starting mass of overall lysate. It has been found that even adding as little as 5% of the purified beta-glucan could have beneficial effects on immune therapy, and on an average in another example, about 15 to 25% could be added. Based on the value of 60% beta-glucan for the lysate, this could correspond to about 150 mg of total beta-glucan per 250 mg of the lysate with other components such as fiber and smaller amounts of protein, vitamins and other components making up the balance. Similar proportions of purified beta-glucan could be added to the dried whole cell Euglena biomass.

The residual media amount can vary, of course, as noted above. The residual media may also be part of the whole cell Euglena gracilis such as manufactured using the process of FIG. 5 and left over from the heterotrophic fermentation process. The starting value of the residual media in the fermentation tank may be independent of the amount of biomass that is growing. The residual media could be glucose or alternative, including vitamin B components and other components such as nitrogen as non-limiting examples. The amount of residual media left over from the heterotrophic fermentation process is not directly proportional to the biomass.

The amount of added vitamin C can vary, but in one embodiment, it is possible to add between about 300 to 500 mg per daily dosage as one typical amount, but smaller amounts are also possible from as low as an added 10 to 20 mg, or 10 to 30, 40 or 50 mg, or 50 to about 70, 80, 90 or 100 mg, or 100 to 250 mg, or about 100 mg with variations from about 10% to 20% from these values. Values may extend up to about 500, 750, or 1,000 mg. This amount may be per capsule or subdivided among several capsules per daily dosage. One example range includes amounts from 50 mg to as high as 1,000 mg or more. Other delivery methods for the composition besides capsules include tablets, powder, lotion, gel, liquid solution, liquid suspension, gummy, multivitamin, health shake, health bar, or a cookie. It is possible to package in a stick pack where the composition may be poured from the stick pack onto food directly.

It has been found via modeling that the linear beta-glucan ratio as in the current composition to vitamin C is preferred at about 1:10 to 1:1. An example range is about 50 mg to 1,000 mg of the Euglena derived beta-glucan, and then the vitamin C may be varied. Although that is a broad range, the range for beta-glucan could be more narrow and 100, 150, 250, 500, 750, and 1,000 mg used with variations of 50 mg and 10 to 20% deviations. About 50 mg to 250 mg, 450 mg, or 500 mg of vitamin C in one example, and in another, up to 750 mg or 1,000 mg of added vitamin C, with variations of 50 mg from these values as non-limiting examples, and allowing a deviation of about 10% to 20%, sometimes 5% to 7.5%. However, it has been determined that a greater amount of beta-glucan relative to vitamin C is advantageous especially with the use of the linear, unbranched beta-1,3-glucan as compared to the prior art use of the yeast based beta-glucan in order to complement the vitamin C.

It is possible to add about 100 mg of the resveratrol with or without the vitamin C. This amount of resveratrol can also vary from about 50 mg to 100 mg, 50 mg to about 150 mg, 50 mg to about 200 mg, 50 mg to about 250 mg, 50 mg to about 300 mg per capsule per daily dosage or with other delivery methods described before for the composition. Amounts may vary 10% to 20% from these values. Higher dosages may be available and used up to 500, 750, or 1,000 mg. It is possible to add further amounts of grape seed or grape seed extract to supply resveratrol.

The amount of humic acid can vary from about 100 mg per capsule or daily dosage and from about 50 mg to about 100 mg, or 50 mg to about 200 mg, 50 to 250 mg, 50 to 300 mg, or up to 500 mg, 750 mg, or 1,000 mg per capsule per daily dosage or other composition delivery method with a 10% to 20% variation and in combination with other components, including the lysate, whole meal, vitamin C, resveratrol, and with or without other components.

As discussed above, the amount of residual media remaining with the lysate or the whole cell can vary, and in one example, the composition may include no more than about 10% of the initial formulation concentration of residual media. As noted above, the ranges vary from as low as 0.5 to 1% to as high as 10% of the initial formulation concentration, and in some examples as noted above, a more likely range is about 1% to 4%, but it may be possible to use about up to 6%, 8%, or 10% as the initial fermentation concentration.

It should be understood that the whole cell Euglena biomass may be substituted for the lysate as described above with similar ranges, percentages and ratios as described above with the beta-glucan and/or whole cell Euglena biomass and may be used with such additional immune response inducing components, such as the vitamin C and other ingredients as discussed above and described in detail below. In an example, the composition may include 10 mg to 1,000 mg of a dried whole cell Euglena biomass derived from a heterotrophic fermentation process and including beta-1,3-glucan comprising at least about 90% linear unbranched beta-1,3-glucan polysaccharide polymers having a molecular weight of 1.2 to 580 kDa and beta-glucan polymer chains having a polymer length about 7.0 to 3,400 glucose monomers. As a dried whole cell Euglena biomass, those values could vary and be greater. About 50 mg to about 1,000 mg of added vitamin C may be included and the weight ratio of the dried whole cell Euglena biomass to the added vitamin C may be in the ratio of 1:10 to 1:1. In an example, the composition may be in the form of the capsule, a tablet, a powder, a lotion, a gel, a stick pack, a liquid solution, a liquid suspension, a gummy, a multivitamin, a health shake, a health bar or a cookie. It should be understood that the powder may be a single use powder such as in a stick pack that may be poured on food as an example since the dried whole cell Euglena biomass may be preferred. Some users prefer what appears to them to be more organic substances and what also appears to be a less processed additive to food, such as the dried whole cell Euglena biomass. The ranges could vary as with the lysate and range from 50 to 100 mg, 50 to 250 mg, 50 to 500 mg, 50 to 750 mg, and 50 to 1,000 mg with other changes in 50 mg increments, and 10% to 20% deviation in each of these values as examples.

About 3,000 mg to 5,000 mg of the whole cell Euglena biomass may be used in a preferred oral dosage form on a daily basis to enhance cardiovascular function. In an example, it is a dried whole cell. The linear, unbranched beta-1,3-glucan polysaccharide polymers may have an average molecular weight of about 140 to 150 kDa. It is also possible to add a purified linear unbranched beta-1,3-glucan to the Euglena biomass such that the whole cell Euglena biomass may comprise at least 85% beta-1,3-glucan with the added purified linear, unbranched beta-1,3-glucan. The dried whole cell Euglena biomass may include residual media remaining from a heterotrophic fermentation process that produced the Euglena biomass, and range up to 10% of the initial fermentation concentration. It should be understood that autotrophically grown Euglena biomass may have a reduced percentage of about 30% or 35%, but can be increased with heterotrophically grown additions.

Besides vitamin C, other components as described below may also be added. The composition with the dried whole cell Euglena biomass and added vitamin C may be contained in a capsule or delivered by other mechanisms as described and the total may be about 100 mg to about 2,000 mg per capsule dosage. Of course, residual media could also be included as left over from the heterotrophic fermentation process that produced the biomass.

The composition may include components inherent to the heterotrophically grown whole cell Euglena and components produced or added during fermentation as part of the growth media. Lipids, proteins, and carotenoids are produced during the fermentation and essentially added that way. Different components include zinc, minerals, vitamins, sugars, amino acids, lipids, proteins and carotenoids. The added vitamin C, the added resveratrol, and added humic acid could be added during fermentation or added to the lysed material or after drying, but are added and separate from what is inherent to the grown algae. They may be added as part of other products, such as grape seed extract in the case of resveratrol or other products. The composition may contain the residual media and added vitamin C or added resveratrol or combination, or added humic acid alone or in combination with added vitamin C, added resveratrol or both, as well as the other components left over from fermentation, since these components are also found beneficial.

The whole cell Euglena biomass can also be left over from the lysis of the cells and contained therein or added back into the lysate, but before drying, or even added in dried form to the final dried lysate. The whole cell Euglena biomass may be separate from a lysed product and used alone, and include residual media and other immune response inducing components. Also, added vitamin C, added resveratrol, and added humic acid alone or in combination can be added during fermentation, and thus, be part of the residual media, or added after lysing but before drying, or after drying. This can also apply to other immune response inducing components as described below. As noted before, it is possible to add the pure paramylon also referred to as the purified beta-glucan produced by the process as shown in FIG. 3 to enrich the glucan concentration in the lysate with amounts as described above and enrich the dried whole cell Euglena biomass.

This lysate formed by the process described above, the purified beta-glucan, and the whole cell biomass incorporate primarily the linear, unbranched beta-1,3-glucan as described above typically at least 90%, but in one example, about 94% as a commercial variety, and could be as high as 99%. It is substantially different from many other commercially available beta-glucans that are derived from yeast, fungi, and seaweed, which all have branched beta-(1,3); (1,6)-glucans from the cell walls of yeast and cereals, for example, oats and barley, and often also include branched beta-1,3-glucans having beta-(1,4)-glycosidic bonds, all forming polysaccharide side chains. There are some suppliers of beta-glucan that publicly state that the side branching may give beta-glucan polymers the ability to stimulate the secretion of cytokines and exhibit high immunomodulation functions. Many suppliers choose to employ branched beta-glucans derived from yeast as their preferred source of beta-glucan since it is easily produced and available and includes extensive side branching. It has been determined, however, that the linear, unbranched beta-1,3-glucan even as low as 60% in the lysate is as effective or outperforms as 85% beta-glucan from yeast as the branched variety. With the added purified beta-glucan added to the current lysate, it becomes even more effective.

The composition may include the other cellular components and residual media that many other commercial suppliers of beta-glucan products remove. The composition may include additional components, including the added vitamin C, the added resveratrol, or added humic acid, or a combination and mixture and other components. It is possible to include the whole cell Euglena with the lysed cellular components that include the linear, unbranched beta-glucan and other added components, but typically it is the purified beta-glucan that may be added to the lysate to increase the percentage of beta-glucan in the product as described above.

There has been a commercial trend of using branched beta-glucans from yeast, microbes, mushrooms or cereals in order to include a full range of branched glucans, including branched beta-1,3-glucans, branched beta-1,4-glucans, and branched beta-1,6-glucans. In some examples, the branched structures are irradiated to form randomly fragmented and branched beta-1,3-glucans, beta-1,4-glucans, and beta-1,6-glucans. As short polymers of 1,3; 1,4; or 1,6 beta-glucans all having extensive side branching with some marketing expectation that these random short segments having the extensive side branching will enhance immune-modulating properties. The branched beta-glucans may be dissolved in a solvent to form a purified beta-glucan composition, completely removing other cellular components and any residual media and not adding additional components.

The Euglena biomass lysate as produced with the process described above with reference to FIG. 4 is heterotrophically grown using a growth media that includes glucose and a nitrogen containing amino acid that increases the paramylon content to much greater levels than the Euglena found in nature and forms a non-natural spherical phenotype that is different from the natural rod-like phenotype found in nature. Also, the described heterotrophically grown Euglena biomass is substantially different than that found in nature since the bulk density is higher, and thus, the whole cell Euglena is heavier and more dense than naturally grown Euglena. As described before in an example, the composition includes the Euglena biomass lysate, and in an example, may include the added purified beta-glucan. It may include the dried whole cell Euglena biomass that has this different phenotype and the residual media. It may include the other components described above, including the added vitamin C, added resveratrol and added humic acid alone or in combination with each other or other components as described, which operate with the beta-glucan in a markedly different manner than what is found in nature.

There is a marked and non-natural cell deformation in Euglenophyta owing to the extraordinary accumulation of paramylon granules when cultured on an enriched medium that is not found in nature. There is a significant shortening and widening of the cells and a change in the pellicle striae to a helicoidal configuration. The bulk density of the cell is also increased greater than that normally found in nature because of the extra glucan. The growth medium used in some of the processes described above includes glucose and one amino acid nitrogen source and may include the added vitamin C, the added resveratrol, and added humic acid alone or in combination and other described components, which aid in the growth of this non-natural phenotype.

Although different sources of vitamin C may be added, one commercial source of vitamin C is Rovimix® Stay-C® 35, which is a stabilized phosphorylated Na/Ca salt of L-ascorbic acid. This ascorbic acid is esterified at position 2 and protects the vitamin C from destruction by oxidation and contains primarily the monophosphate ester of L-ascorbic acid with small quantities of diphosphate ester and traces of triphosphate ester. The amounts and percentages as described above for this example vitamin C may be used. The added vitamin C enhances the function of the beta-glucan, and especially with the lysate of linear, unbranched beta-1,3-glucan and the purified beta-glucan. It can be added with the whole cell Euglena biomass, however. The added vitamin C together with the residual media operates with the lysate or purified beta-glucan or combination of both.

Resveratrol may be added in an example, and is not naturally found in Euglena and may be added alone or in combination with vitamin C. The resveratrol and vitamin C added with the lysate or purified beta-glucan allows all three to operate synergistically in an enhanced function with immunomodulation function. The lysate and purified beta-glucan is particularly beneficial since the added vitamin C and added resveratrol in combination with the other components, including the linear, unbranched beta-1,3-glucan is made available faster when orally ingested such as in a non-limiting capsule form since it is in free form. Also, the incorporation of the dried whole cell Euglena biomass within the composition and in the non-limiting capsule form permits a slower breakdown of the cell wall pellicle once ingested, and permits those components, including unlysed Euglena and the vitamin C as part of Euglena and not the added portion, to enter the body's system more slowly, operating in a delayed manner similar to a time-delay medication. Other delivery methods as described may be used.

When a lysate is administered to an animal or human subject, the compounds may not be as bound and the lysate may operate as a superior delivery method for the glucan and added vitamin C or other components when applied, and alternatively or in combination, the added resveratrol and other residual media, including humic acid or other components that may be added as described and may increase synergy. The lysate and components such as from the residual media are more available to the body and more quickly since they are free. Having the dried whole cell Euglena biomass where any components are less free is also advantageous since the pellicle must be broken down, and thus, any smaller amounts of vitamin C and other components, including linear, unbranched beta-1,3-glucans, could also still be available at a later time as compared to that which is already free and part of the lysate or residual media. The composition thus includes components that operate similar to a timed medicine delivery system, and may be formulated into a single dose capsule or other delivery mechanism in the amount of about 50 mg to about 2,000 per capsule dosage or other delivery mechanism, and with a capsule or tablet, depending on the compaction, but can vary from about 100 mg to about 500 or 600 mg, and up to 1,000, 1,500 or 2,000 mg, or from 100 to 250 mg, up to 500 mg, up to 1,000, 1,500 mg, 1,800 mg, or 2,000 mg to 2,500 mg and any combination with variations of about 10% to 20%. One dosage may be 400 to 600 mg with larger amounts divided among multiple capsules, and depending on compacting that may be accomplished, including roller compacting.

The humic acid may be added either alone or in combination with the vitamin C, resveratrol and also added with whole cell algae biomass as described above. Humic acid is not found in the Euglena algae or other sources of beta-glucan since it is usually produced by the biodegradation of dead organic matter, and it may be used to treat heart disease and possibly aid cancer patients and may be a positive benefit to aid in digestion, boosting nutrient absorption, gut health, immunity, cognitive functioning, energy levels and protect from infections, viruses, yeasts, and fungus while boosting skin health. The humic acid may also work to enhance the effect of the beta-glucan in a synergistic manner.

It has been determined that the combination of humic acid and beta-1,3-glucan enhances the effect of each other. It is believed that the oral supplementation helps reduce levels of ALT and AST in the serum of tested animals and the combination of the humic acid and beta-glucan are very active. The prophylactic effects may decrease liver damage and this efficacy may be caused by the strong potentiating action of the antioxidant protective system, supported by protection of the GSH levels, where the glucan-humic acid combination is enhanced to mop up free radicals and limit their destructive effect. Folic acid may also be added in amounts similar to the amounts of humic acid. One aspect is that they are of lower molecular mass and more biologically active and may enhance some bioavailability of the beta-glucan or synergy.

The dried whole cell Euglena biomass may have the residual media remaining from a heterotrophic fermentation process that produced the Euglena biomass. Besides the vitamin C or humic acid, the composition may include an added immune response inducing component, such as one or more of vitamin C, Echinacea, aloe, golden seal, ginseng, garlic, bell peppers, ginger, tumeric, gingko biloba, cat's claw, ganoderma, astragalus, humic or fulvic acids, resveratrol or other polyphenols, broccoli, spinach, yogurt, almonds, honey, green tea, papaya, kiwi, poultry, shrimp, sunflower, vitamin D, mushrooms, pumpkin, cinnamon, parsnips, grapes, sweet potatoes, milk, orange juice, rice, carotenoids, figs, glutamine, arginine, an omega-3 fatty acid, vitamin A, vitamin E, selenium, zinc, a probiotic, corn, soy or corn or soy derivatives, including dried distiller grains. Additionally, the composition may be in the form of a capsule, a tablet, a powder, a lotion, a gel, a stick pack, a liquid solution, a liquid suspension, a gummy, a multivitamin, a health shake, a health bar or a cookie.

The beta-glucan as a lysate or whole cell biomass may have added Echinacea and the beta-glucan and Echinacea may operate together. The beta-glucan may rejuvenate cells by increasing the production of macrophages and amplify the rate of B-lymphocytes and reduced T-cells while operating as a better antioxidant in combination with the Echinacea. Dosages may be 1,000 mg and combinations of around 500 mg for each of the beta-glucan as a lysate or whole cell and Echinacea such as a purified Echinacea purfurea. There is evidence the beta-glucan and Echinacea operate together to help relieve upper respiratory tract symptoms. Other ranges and combinations may be used.

An aloe, such as aloe vera, has numerous active biological compounds and may work in combination with the beta-glucan, and more particularly, the lysate as formed and enhance the purified beta-glucan or whole cell biomass. In some studies, it is shown that the combination may stimulate both cellular and humoral immune responses and increase platelet counts.

The amount of the beta-glucan and aloe can vary, and in one example, a 1:1 ratio such as an equal amount of each with 50 mg of beta-glucan and 50 mg of aloe may be used and combined with a cellulose filler for a capsule such as microcrystalline cellulose. There have been some commercial embodiments using the branched form, but none known using the linear unbranched form of beta-glucan. This may be beneficial since the aloe may include bioactive maloyl glucans that work in combination with the linear unbranched beta-glucans. The lesser amount of aloe may be used with greater amounts of beta-glucan such as 250 mg of the lysate combined with about 50 mg of aloe and with a percentage deviation of about 10% to 20%.

Golden seal combined with the beta-glucan may work for immune functions, and may also help control muscle spasms and simulate the heart and increase blood pressure for those with lower blood pressure problems and sometimes treat gastrointestinal disorders. In fact, it may work in combination with the beta-glucan because the golden seal may drop the level of intestinal pathogens or impact those pathogens and other intestinal components so that the beta-glucan will be more bioavailable. In an example, about 30 to 50 or 60 mg of golden seal may be combined with the beta-glucan at about 200 to 1,000 mg in an example, or about 250 to 600 mg to allow the beta-glucan to be absorbed better. Ginger root or other varieties of ginger may be added to improve the body's ability to absorb the beta-glucan in an example, and in an example, 30 mg to about 60 mg of ginger may be added in an example, and up to about 45 mg.

It is also possible to use Panax Ginseng or other varieties of ginseng. It is also possible to use Ginkgo Biloba. One or both of the Ginkgo Biloba or Panax Ginseng may be combined with the beta-glucan. About 320 mg to about 960 mg of a combination of ginseng and Ginkgo Biloba can be added with different ratios. One known ratio is at about 3:5 ratio or thereabouts. Other combinations may be used and found beneficial. The amount of ginseng used in combination with the beta-glucan, whether the lysate or whole cell biomass, may vary from as little as 150 mg to as much as 600 mg and the Ginkgo Biloba can vary from as low as 50 mg to as much as 200 mg or 250 mg and any variations therebetween with variations of about 10% to 20%. The Ginkgo Biloba and ginseng can be added alone or in combination.

In an example, the Ginkgo Biloba that may be used in the current composition may be a leaf extract having 24% glycosides and 6% terpenes. The Ginkgo Biloba could be a standard extract or it could be an extract specifically prepared for use with other components, including the beta-glucan. The Ginkgo Biloba extract may be formed as a concentrate and obtained from its leaves whether dried or fresh and prepared in one example using an acetone-water solution.

Ginkgolide B terpene is a potent antagonist against platelet activity factor and inhibits platelet aggregation, fibrinolysis, and thrombin activity and for that reason, care is often given when employing the Ginkgo biloba extract, especially in combination with other components. In combination with beta-glucan, there may be enhanced bioavailability and efficacy.

There may be some protection of neurons from oxidative stress, e.g., apoptosis and also reduce the toxic effects of cerebral ischemia. It may also help reduce the production of arachidonic acid as a byproduct of lipid metabolism. It may have antioxidant function.

One standardized Ginkgo Biloba extract is concentrated in a ratio of 1 part extract to 50 part dried leaves and contains 24% flavone glycosides (quercetin, kaempferol and isorhamnetin) and 6% terpene lactones (2.8-3.4% Ginkgolides A, B and C, and 2.6-3.2% bilobalide). In France and Germany, the Ginkgo biloba extract has been prescribed for tinnitus, headache, dizziness, depression, anxiety, confusion, problems with memory and concentration, and other conditions.

Ginkgo Biloba extract alone helps age-associated cognitive decline and slow the progression of neurodegenerative diseases associated with dementia such as Alzheimer's disease. Beta-glucan operates as an immune function modulator and with Ginkgo Biloba, it may enhance the effects of the Ginkgo Biloba antioxidant and anti-inflammatory properties, and preserve mitochondria function and increase ATP production, inhibit 3 amyloid formation, reduce neuron apoptosis, and enhance cholinergic transmission.

Ginseng refers to species of the Panax genus of the Araliaceae plant family. Ginseng effects may become more apparent when a person's resistance is diminished and the beta-glucan may enhance the effect and be helpful when a person requires extra demands in mind and body. Some evidence shows that individual ginsenosides have anti-inflammatory effects in vivo and in vitro and possess anti-mutagenic and DNA protective properties. With the beta-glucan, this may be helpful.

The ginsenoside saponins may contribute to the beta-glucan and can be classified into three groups based on chemical structure: 1) the Panaxadiol group (Rb1, Rb2, Rb3, Rc etc.); 2) the Panaxatriol group (Re, Rf, Rg1, Rg2, Rh1); and 3) the oleanolic acid group (e.g. Ro).

It is also possible to enhance the effectiveness of the beta-glucan that shift inflammatory profiles to a Th1 type to enhance resistance against bacterial and parasitic infections and possibly include several polyunsaturated fatty acids such as omega-3 fatty acids from fish oils, including EPA and DHA and possibly plant-derived N-3 fatty acid alpha linolenic acid. The fatty acids may work in combination with the beta-glucan to activate toll-like receptors, and thus, the inflammatory pathway. Use of fatty acids, and more particularly, EPA and DHA and ALA and/or LA, may improve intestinal barrier function while the Ginkgo Biloba could lower the nuclear factor Kappa B and activator protein 1 due possibly to the higher content of polyphenols.

Thus, it is possible to apply the Ginkgo Biloba with the beta-glucan and other polyphenols, including the resveratrol or other grape seed extracts. Of course, it is also known that the beta-glucan is recognized and taken up by immune cells such as macrophages or dendritic cells via beta-glucan receptors (dectin-1\TLR-2) on the cell membrane. The immune cells may regard them as “pathogen-associated molecules” and elicit an activated immune response. Ingredients such as the saponins may potentiate the immuno-stimulatory effects of beta-glucan and enhance the efficacy.

Garlic may be supplemented from as low as 200 or 300 mg to intermediate ranges and as high as 600 mg to 700 mg. As noted before, greater amounts of the lysate or whole cell Euglena biomass may be used for cardiovascular purposes, including up to 3,000 to 5,000 mg per day dosage range distributed among various capsules. This may have an effect on serum LDL cholesterol concentrations and operate to lower them. Since the viscosity in the gastrointestinal tract may be varied by supplementation with other components, the garlic also may operate with the beta-glucan. Soluble fibers may be added to help increase the binding of bile acids in the intestinal lumen leading to decrease and enterohepatic circulation of bile acids and increase in the hepatic conversion of cholesterol for bile acids. Oats may be added and other components. This would give some increase in the branched beta-glucan. Also, the oats have other advantages for intestinal motility.

Soy protein may be added to increased levels of genistein and daidzein and red clover added to contain higher amounts of biochanina and formononetin, such that the biochanina and formononetin can be converted to genistein and daidzein, respectively. The soy protein or red clover may be added at about 30 to 100 mg, and in another example, 40 to 80 mg per daily dosage. Plant sterols or stanols may also be added that include campesterol, beta-sitosterol, and stigmasterol. The garlic may include the allicin as the active lipid-lowering compound and the garlic clove may allow the enzyme alliinase to operate more effectively. The tocopherols and tocotrienols as sub-groups of the vitamin E family may be incorporated with the beta-glucan. It is possible that with ginger the beta-glucan combination may operate for a skin care application.

Ginger may be applied in a range from about 10 mg to about 60 mg, and from about 100 mg to 200 mg, and up to 300 mg to 400 mg with ranges therebetween and may have efficacy with the beta-glucan as described above. The different oils in ginger may be advantageous such as the zingerone, shogaols, and gingerols. It stimulates the production of saliva as a sialagogue action to make swallowing easier and helps alleviate some nausea and vomiting especially for those under chemotherapy, and thus, may be a good additive with the beta-glucan when used for immune function. Sometimes ginger may be combined with turmeric to reduce inflammation and help with to stomach issues where the ginger may reduce symptoms of nausea and vomiting and turmeric may reduce symptoms of indigestion such as bloating and gas. Ginger and turmeric can have similar amounts with the beta-glucan and the ginger and the turmeric may range from 100 or 200 mg to 300 or 400 mg.

It is also possible to apply vitamin D with the beta-glucan and add selenium with vitamin D. Vitamin D may be added in the amount of about 1.0 ug to 15 ug and preferably about up to 10 ug. Vitamin D may be formulated as a vitamin D3 in an example since it may aid in the normal immune function. However, vitamin D2 or a combination with D3 can be used. Different mushrooms may be added including ganoderma.

The composition may include other mushroom additives that also operate with beneficial effects on the immune system, cardiovascular system and prostate gland. The composition may stimulate various types of white blood cell production and increase antioxidant activity in plasma. The composition may have some blood-thinning properties to inhibit platelet aggregation and may also dilate arteries. The composition may produce different triterpenes as ganoderic acids, including beta-glucan and can be provided as a ground mushroom, such as reishi mushroom, and formulated as a capsule with the beta-glucan such that the ganoderma is about 100 mg to about 600 mg or lower, including 200, 300, or 400 mg with different amounts of lysate or combination as described above. Other mushroom varieties may be used.

It is possible to use astragalus, which is often used as a traditional Chinese medicine, but may be combined with the beta-glucan as a lysate or whole cell biomass. The astragalus may be varied in combination with the beta-glucan and can vary from as little as 100 mg to as much as 1,200 mg with a combination of about 300 to 500 mg. The polysaccharide, triterpene, which may be part of the astragalus and various flavonoid fractions may operate and be credited with immune-regulating actions. The astragalus may include beta-glucan and astragalin and other saponins. The immune polysaccharides in astragalus are usually of higher molecular weight and not easily absorbed from the intestines, and thus, may trigger some immune responses on the intestinal mucosa and microbiota. However, because of the higher molecular weight, other components may be used to aid in bioavailability and the beta-glucan may help in this regard.

Mushrooms may provide some beta 1,3:1,6 D-glucan that are branched and may work in combination with different functions with the linear beta-glucan as described above. They may work together to activate leukocytes that depend on the different structural characteristics of the beta-glucans and enhance each other.

Papaya may be an additional source of beta-glucan. It includes an number of phytochemicals, including carotenoids and polyphenols as well as benzyl isothiocyanates and benzyl glucosinates. The extract of various chemicals may be used or a papaya enzyme used as a digestive aid that may help with the bioavailability of the beta-glucan. In an example, 1 to 5 mg of papaya fruit may be added with different enzymes or much larger amounts of papaya used. Twenty to 30 mg of papaya as the fruit may be combined with different enzymes such as papain, protease, or amylase.

Kiwi may also be added and it is advantageous to possibly allow the beta-glucan and kiwi to operate synergistically together and may act as a prebiotic and added as a powder of anywhere from 50 or 100 to 150 or 200 or 250 mg and added as a fruit extract from 100 or 200 mg to about 500 to 600 mg or up to 650 to 700 mg. One advantage of kiwi fruit is the negligible protein and fat, but it is particularly rich in vitamin C and vitamin K and has a moderate amount of vitamin E. The seed oil derived from kiwi fruit may contain an average of 62% alpha-linolenic acid (ALA) and the pulp may contain carotenoids such as pro-vitamin A, beta-carotene, lutein, and zeaxanthin. Parsnips may be added in combination with vitamin C or folate or other vitamins. It may be a good source of fiber and improve heart health. It has been used for digestion problems and thus may enhance the bioavailability of the beta-glucan. It also has been used for fluid retention disorders and thus helps on the bioavailability of the beta-glucan.

Cinnamon may help lower blood sugar that may help on the bioavailability of the beta-glucans. Cinnamon may also have an antibacterial effect to aid bioavailability of the beta-glucans. Small amounts such as 40 to 60 mg, or 40 to 100 mg may be used, but larger amounts of cinnamon, e.g., over 250 mg, and up to 250 mg to 500 mg, may be added.

As noted before, cat's claw is a common name for several plants and may be used in combination with the beta-glucan. One woody vine is uncaria tomentosa and also uncaria guianensis. It contains polyphenols and caffeine that are advantageous. These components may be extracted and used. It may be combined with the beta-glucan in different amounts at about 40 to 60 mg for the cat's claw, or 100 mg, and with green tea amounts of 100 or 200 mg up to 500, 600 and 700 mg or more.

A number of combinations of these different components may also be used in combination with the beta-glucan such as 20 to 50 mg of vitamin C, and 20 to 40 IU of vitamin E as D-alpha tocopherols and a green tea at about 40% extract at about 150 to 300 mg, and in an example, about 200 mg, and the cat's claw at about 10 to 30 mg and at an average 20 mg, and the same for garlic powder. The ginseng could be about 10 to 30 mg while higher amounts of grape seed extract at around 50 to 150 mg could be used. The ranges of each of these components may vary 10%, 20% and 30% and ranges therebetween. Other components such as selenium, cucurmin, lycopene, and other components, including an olive leaf extract may be added. Spinach may contain thylakoids and help in the satiety cascade for better eating function and digestion and thus aid in more beneficial use and absorption of beta-glucan.

Other components of leafy vegetables including broccoli may be advantageous. The beta-glucan may also boost the nutritional value of yogurts, including low-fat yogurt. The beta-glucan can be a source of fiber, and most advantageously, a prebiotic that affects the yogurt-mix qualities with a very highly purified such as 90% to 95% pure beta-glucan added at about 0.1% to 0.3%, corresponding to about 0.3 grams (300 mg) of beta-glucan per 100 grams of yogurt mix. This range has been found advantageous without affecting the consistency adversely. The ranges can vary 10% or 20% from those values. The beta-glucan addition, for example, as a prebiotic, to a probiotic containing yogurt may suppress proteolytic activity. It is possible to add two prebiotics, such as the composition of the beta-glucan and an oligosaccharide, such as a galactooligosaccharide that is a fiber such as found in breast milk, and have fewer side effects than insulin.

Bee honey or other sources of honey can be applied as an additional source of energy and glucose with beta-glucan. An advantage of honey is the whole cell beta-glucan biomass may be added to the honey for some commercial uses that are more acceptable to consumers. The lysate can also be added, but some consumers desire the whole cell beta-glucan. Honey has viscous properties and water may be added to it to allow the honey to flow more easily. Honey may absorb moisture from the air and some fermentation of honey may affect the beta-glucan. The amount of honey may vary, but a 1:10 ratio of beta-glucan relative to the honey may be used and up to a 1:1 ratio in a non-limiting example. It is possible to add the whole cell beta-glucan biomass or lysate or a combination of whole cell biomass and lysate and/or purified beta-glucan to milk or orange juice. Milk contains additional proteins such as arranged in casein micelles similar to a surfactant micelle bonded with the help of nanometer-scale particles of calcium phosphate that may work in synergy with beta-glucan or help in bioavailability or absorption. Milk also contains some enzymes that may affect the synergy and/or bioavailability of the beta-glucan in a positive manner. The composition may also be added to an infant formula.

Beta-glucan whether the whole cell biomass or lysate form added to orange juice may work in synergy with various components, including some of the vitamins in orange juice. Also the juices contain flavonoids that have health benefits. The added vitamin C could be from part of the orange juice and subsequent amounts of vitamin C added.

Common fig may be used with the whole cell beta-glucan or the lysate. One aspect of the figs is they contain diverse phytochemicals, including polyphenols, including gallic acid, chlorogenic acid, syringic acid, catechin and epicatechin and rutin. As noted before, carotenoids may be added such as xanthophylls that contain oxygen and carotenes that are the hydrocarbons and contain no oxygen. Euglena gracilis and especially some mutant varieties may contain phytoene and other components and especially as part of the lysate. The added carotenoids may have some synergy or other benefits with these components.

Other immune response inducing components may be added, including amino acids such as glutamine that is a trophic for immune cells and circumvents oxidant stress and arginine that operates as a substrate for synthesis of nitric oxide and enhancement of Th cells. Omega-3 polyunsaturated fatty acids may operate as an anti-inflammatory and vitamin A may operate to regulate Th1\Th2 balance while vitamin E may circumvent oxidant stresses and operate as an anti-inflammatory. Selenium may stimulate cell-mediated immune responses. Zinc may have a similar function. Added nucleotides may also stimulate cell-mediated immune responses. Probiotics may stimulate IL-12\IL-10 production, and in an example, probiotics may include peptidoglycan and lipoteichoic acids. CpG oligonucleotides may operate as an anti-inflammatory.

Probiotics may also stabilize the intestinal microflora and normalize intestinal microflora that could lead to a modulation of a host immune system. Lactic acid bacteria may operate as probiotics and be recognized by specific receptors on the surface of phagocytic cells. The vitamins and minerals as indicated above and fatty acids such as the omega-3 polyunsaturated fatty acids may affect cellular functions and help preserve the cell membrane and regulate gene expression after being incorporated into lymphocytes.

Glutamine may improve nitrogen retention and lower the incidence of bacteremia. The supplementation of a glutamine-enriched diet with the glucan may help recover immune functions. The glutamine acts as a precursor for glutathione and helps circumvent the oxidant stress and improve cell-mediated immunity. The arginine may operate as a substrate and help synthesize nitric oxide and improve the helper T-cell numbers, while its combination with the omega-3 polyunsaturated fatty acids may help restore the DTH (delayed-type hypersensitivity) and decrease infection rates in some cancer patients. It is also possible to add feeds containing corn, soy, or corn or soy derivatives, including dried distiller grains.

The dosage range such as in a capsule or tablet or other delivery mechanism for a combination of these different components, including the Euglena lysate or whole cell Euglena biomass may be as small as 500 mg as noted before, but range up to 1,000, 1,500, 2,000 or 2,500 mg and any other combination thereof and, of course, the amounts may depend on how compressed the composition is for delivery and end use requirements.

The whole cell Euglena biomass or the Euglena lysate or any combinations may be added to an animal feed product. An example is an animal feed product formulated to feed domesticated animals such as dogs and cats, and in yet another example, an animal feed product having specific nutritional requirements such as for a mouse diet, or meet general nutritional requirements for a generic animal feed. The animal feed product could be formulated as a specific diet for swine or poultry. Different animal feed products having the added whole cell Euglena biomass or added Euglena lysate could also include purified beta-glucan in addition to the whole cell biomass, lysate or combination. In addition, the ranges of ingredients for specific feeds may vary depending on the growth stage of a particular animal as explained below.

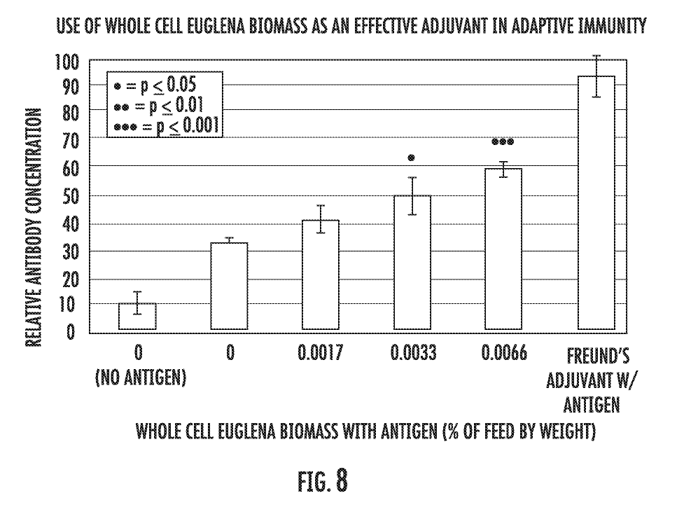

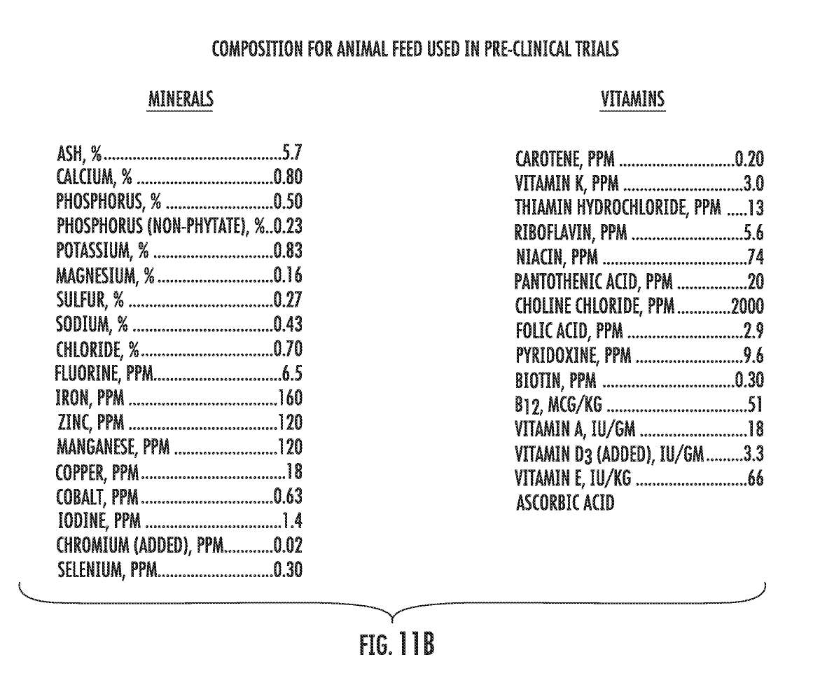

Pre-clinical trials were performed using the whole cell Euglena biomass added into an animal feed product, which in this example was formulated as a mouse diet in order to test the efficacy of an animal feed product having an added whole cell Euglena biomass. The results of these pre-clinical trials are shown in FIGS. 8 through 10. The ingredient listing for the animal feed product into which the whole cell Euglena biomass was added are shown in FIGS. 11A and 11B. In this example, there was some residual media remaining as explained above.

These pre-clinical trials demonstrated how particular immune parameters behaved when the animal feed product that includes the whole cell Euglena biomass was given orally to mice. These studies independently supported the use of whole cell Euglena biomass to increase an animals' level of immunity via immunopotentiation, which accentuates or enhances the immune system to recognize and help protect the body from foreign invaders. The whole cell Euglena biomass as described above and that includes in an example some residual media when added in small amounts to the animal feed product can improve both branches of immunity, the specific (adaptive) and non-specific (innate). This study produced data from four scientifically well-accepted analyses, testing the adaptive and innate immune system responses:

1) Antibody production (adaptive) as a quantification of pathogen recognizers that improve the body's efficiency in halting future infection;

2) Phagocytosis (innate) as a measurement of how effective the immune cells are at engulfing and eventually destroying foreign pathogens;

3) Natural Killer Cell activation (innate) as a determination of the expected immune cell response to viral infection and tumor formation; and

4) Interleukin-2 (innate) as an evaluation of a signaling compound produced by the body as a communication mediator to immune cells.

These studies were conducted with three mice per condition, at three different concentrations of whole cell Euglena biomass added to the animal feed product and under controlled environments. Variability is shown using the bar charts, indicating the standard error on the figures, while p-values are annotated with diamonds to indicate the level of confidence in the result. As an estimate, the feed percentages tested in the study targeted low inclusion rates ranging from 0.034 to 0.132 pounds of whole cell Euglena biomass per 2,000 pounds of animal feed product, corresponding to 0.0017 percent whole cell Euglena biomass to the animal feed product and up to 0.0066 percent whole cell Euglena biomass to the animal feed product. An intermediate 0.0033 percent was tested also to support dose dependency. It should be understood, it is believed similar percentages could be used effectively for the Euglena lysate and the results would be similar. An example range for a commercial product could be 0.0001 to 1.0 percent (weight/weight) of the whole cell Euglena biomass or lysate to the total feed and a more preferred range of about 0.0001 to 0.001 percent, or about 0.0001 to 0.01 percent, and as high as 0.124 percent, and more preferably about 0.0001 to 0.0124 percent, and in another example, about 0.001 to 0.01 percent. Depending on the feed, it is possible to reach a higher percentage up to 0.0125 percent and up to 0.014 percent, and even 0.025, 0.05, 0.075, and 0.1 percent in some non-limiting examples.

FIG. 8 is an example bar chart for the evaluation of the formation of antibodies using ovalbumin as an antigen. Mice were injected twice (two weeks apart) with 100 μg of ovalbumin and the serum was collected seven days after the last injection. The levels of specific antibodies against ovalbumin were detected by ELISA. As a positive control, the combination of ovalbumin and Freund's adjuvant was compared. In all cases, supplementation of the animal feed product with the whole cell Euglena biomass increased antibody production. The tested highest dose increased antibody production by 84% compared to the control with no adjuvant.

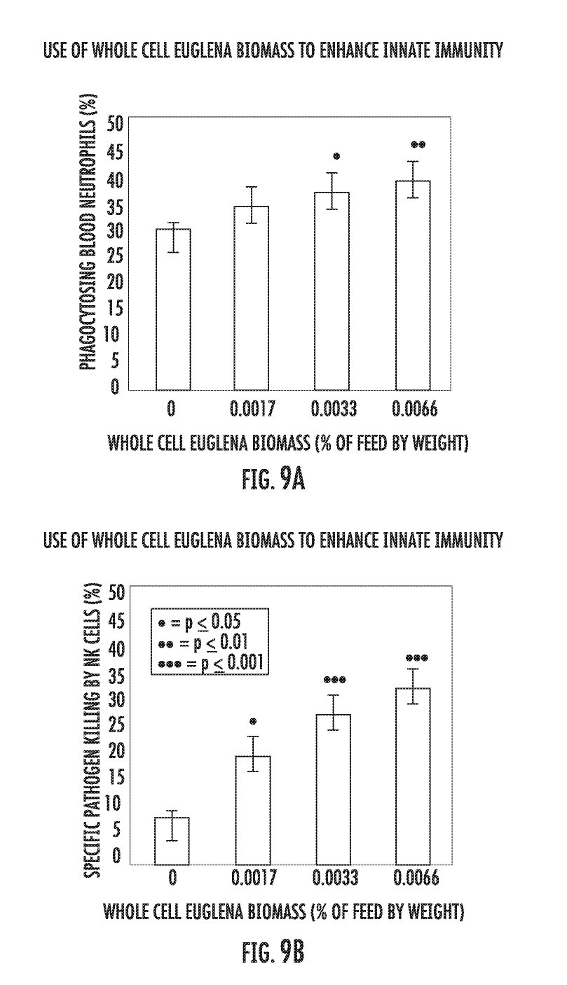

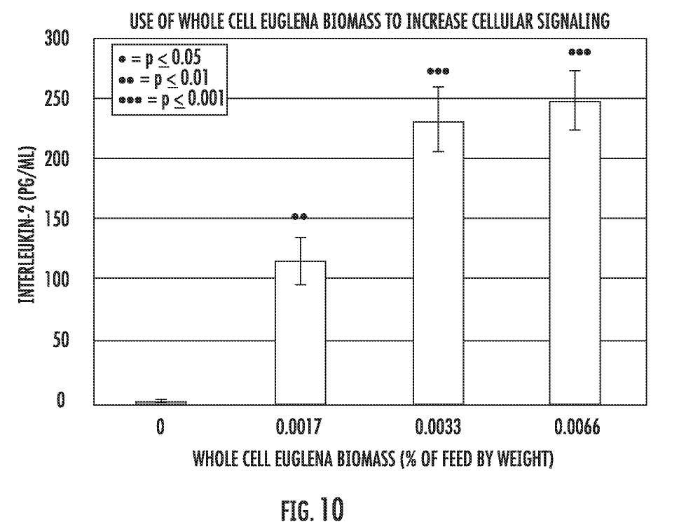

FIG. 9A is a bar chart showing the results of the pre-clinical trial of the phagocytic response where a standardized micro-method with polymeric HEMA microspheres was utilized. Cells used in this study were peripheral blood neutrophils collected at the end of supplementation. To determine the effect on Natural Killer activity, on the other hand, the spleens of mice were minced and cells were purified, washed and resuspended in buffer. Viability was tested by Trypan Blue exclusion and suspensions containing >95% viability were selected. The cytotoxic activity of the cells was determined using the CytoTox 96 Non-Radioactive Cytotoxicity Assay. Results are shown in FIG. 9B. In each case a dose-dependent response was clear. Phagocytosis and Natural Killer cell activities were shown to increase up to 31% and 245%, respectively.

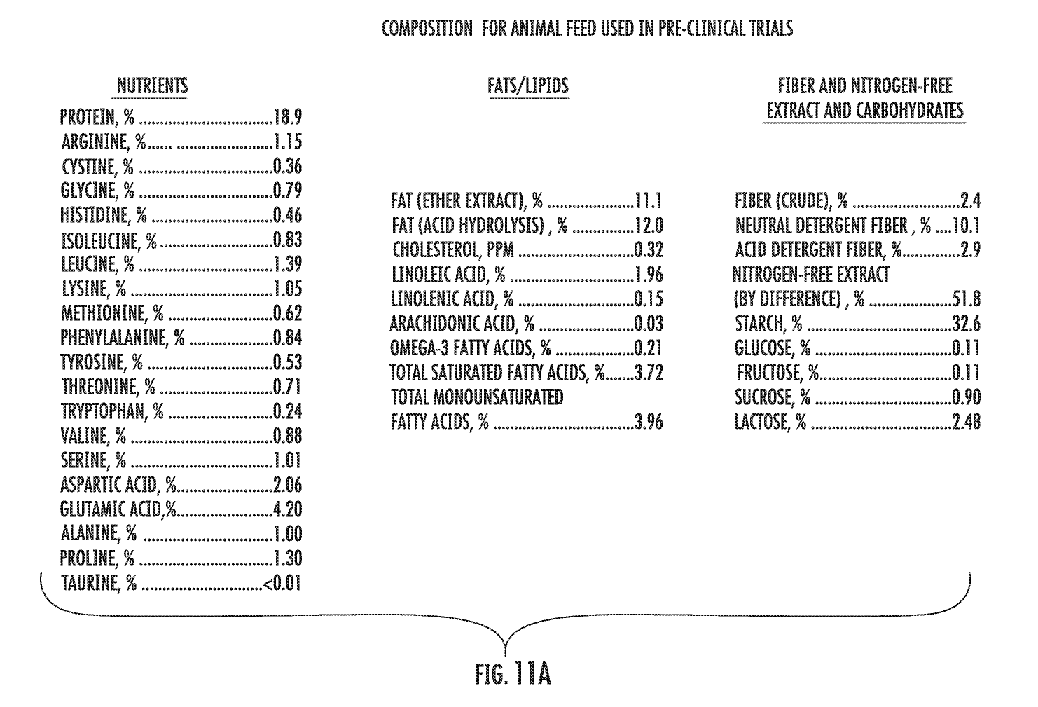

For analysis of the effect on cellular signaling, purified spleen cells from mice were added into wells of a 24-well tissue culture plate. After addition of 1 μg of Concanavalin A, cells were incubated for 48 hours in a humidified incubator. At the endpoint of incubation, supernatants were collected, clarified and tested for the presence of IL-2 using a Quantikine mouse kit. The results are shown in FIG. 10 and show that when supplemented with whole cell Euglena biomass containing beta-1,3-glucan, the near zero basal levels of IL-2 are significantly increased (>150 times) which is expected to enhance the immune communication in the body.

The animal feed product used in this pre-clinical trial was formulated specifically as a mouse diet composition that supports production, growth and maintenance. In an example, it contained about 11% fat and was helpful for post-partum matings but the proportion of ingredients could be similar or modified for different animal feed products. In this example, the delivery mechanism could be a meal as a ground pellet or an oval pellet, such as ⅜ inch by ⅝ inch by 1 inch in length. This animal feed product may contain not less than 17% crude protein, not less than 11% crude fat, not more than 3% crude fiber, not more than 6.5% ash, and not more than 12% moisture. In another example, these values could be 5% or 10% from the stated values. Usually adult mice will eat up to 5 grams of pelleted ration daily and as much as 8 grams per day. This example diet used in the trials included a number of ingredients: whole wheat, dehulled soybean meal, ground corn, wheat germ, brewers dried yeast, porcine animal fat preserved with BHA and BHT, condensed whey, porcine animal fat preserved with BHA and citric acid, condensed whey solubles, calcium carbonate, salt, dried whey protein concentrate, soybean oil, mono and diglycerides of edible fats, DL-methionine, dicalcium phosphate, menadione dimethylpyrimidinol bisulfite (source of vitamin K), choline chloride, pyridoxine hydrochloride, cholecalciferol, vitamin A acetate, biotin, ell-alpha tocopheryl acetate (form of vitamin E), folic acid, vitamin B12 supplement, thiamine mononitrate, ferrous sulfate, calcium pantothenate, nicotinic acid, riboflavin supplement, manganous oxide, zinc oxide, ferrous carbonate, copper sulfate, zinc sulfate, calcium iodate, cobalt carbonate, and sodium selenite.

FIGS. 11A and 11B are charts showing the different ingredients used in this example animal feed product for the pre-clinical trials. FIG. 11A shows the different nutrients, fats, fiber, nitrogen pre-extract, and carbohydrates, and FIG. 11B shows minerals and vitamins. As an example, the composition may include 85.4% total digestible nutrients and 4.74 kcal/gm gross energy and 3.83 kcal/gm physiological fuel value and 3.59 kcal/gm of metabolizable energy. The calories can be provided by about 19.752% protein, 26.101% fat (ether extract) and 54.148% carbohydrates. These values can also range from about 5% to 10% of stated values. The variations in the values for the composition as described above in FIGS. 11A and 11B can vary from 5% to as much as 10%, 15% or 20% from stated values.

These pre-clinical trial results show that much smaller proportions of the whole cell Euglena biomass may be incorporated with an animal feed product and still produce advantageous results to increase immunity levels in the animal, and thus, correspondingly if administered to humans. This remarkable and unexpected discovery is substantially different than the substantially greater amounts required by other researchers in the field. For example, the higher beta-glucan dosing such as described in Table 4 of U.S. Patent Publication No. 2013/0216586 to LeBrun et al. shows a preferred percentage as a daily feed where beta-glucan is 0.10% of the feed, and ranges from a high of 1.0% to a minimum of 0.01%. LeBrun et al. gives an example composition used to feed swine. This is to be compared to what the present inventors have determined in their trials using whole cell Euglena biomass (and lysate in some cases). That preferred range they discovered is much lower at 0.001% to 0.01% or 0.0124% of the animal feed product and could be as low as 0.0001% as explained above.

Another prior animal feed product example uses even larger amounts of algae to support an algal-based animal feed product and is described by U.S. Patent Publication No. 2015/0201649 to Lei, which discloses a product having one or more grains in an amount totaling 50 to 70% w/w of the composition, a non-algal protein source of about 15 to 30% w/w of the composition, and a very high algae amount totaling 3 to 15% w/w of the composition. Other ingredients include an oil heterologous to the algae of about 0.5 to 15% w/w, and an inorganic phosphate source, sodium source, and one or more amino acids. Another prior example shows in Table 1 of U.S. Patent Publication No. 2015/0181909 a preferred higher amount of Euglena algal meal dosing of a minimum requirement of about 0.0125% up to 0.05% for animal growth. Contrary to this teaching, the present inventors have found for increasing immunity levels, a lower range is possible.

The inventors of the current invention have found that much lower amounts of the added whole cell Euglena biomass or the Euglena lysate produced using the techniques described above relative to FIGS. 1-7 have efficacy and advantageous results to increase immunity levels in an animal as also shown in the pre-clinical trial results described above.

It should also be understood that the animal feed product having the added whole cell Euglena biomass or lysate as used in the current invention may contain corn, soy, corn or soy derivatives or byproducts or grains such as dried distiller grains as majority components. Different grains may include maize, wheat, rice, sorghum, oats, potato, sweet potato, cassava, DDGS and combinations, and may be supplemented by proteins such as soybean, fish meal, cottonseed meal, rapeseed meal, meat meal, plasma protein, blood meal and combinations. It could include different oils such as corn oil. It could include other animal feed components as grains or derivatives, including dry rolled grains, alfalfa hay, dehulled soybean meal, vitamin/mineral premix, corn ground, whole cottonseed, cottonseed hulls, cottonseed meal, and fish meal. Other corn derivatives could include: alpha tocopherol, ascorbic acid, baking powder, calcium stearate, caramel, cellulose, citric acid, citrus cloud emulsion, corn flour, corn oil, cornstarch, corn syrup, dextrin, dextrose (glucose), diglycerides, ethylene, ethyl acetate, ethyl lactate, fibersol-2, fructose, fumaric acid, gluten, golden syrup, high fructose corn syrup, inositol, invert sugar, malt, maltodextrin, margarine, monoglycerides, monosodium glutamate (MSG), polydextrose, saccharin, semolina, sorbic acid, sorbitol, starch, sucrose, treacle, vanilla extract, white vinegar xanthan gum, xylitol and zein.

In one example, it should be understood that the animal feed product as the composition includes feeds containing corn, soy, or corn or soy derivatives, or grains or other components, and could be about 40% to 95% w/w of the composition, but can range from 45% to 75%, and in yet another example, and could be about 50% to 70% w/w of the composition. A protein source could be added of about 10% to 40% w/w of the composition, and in another example, about 15% to 30% w/w of the composition. The sources from which the protein could vary as described herein.

Even though the animal feed product overall can vary in its ingredients such as the grains and protein source, other components could range from 1% to 20% w/w of the total composition and could include various minerals, nutrients and oils such as lipids. Added lysine could be an important amino acid for some animals. The animal feed product as the composition with whole cell Euglena biomass, lysate or both could be delivered as pellets or powder depending on the desired delivery mechanism. Other ingredients as described above could be added, including those components for added immune response.

The composition ingredients for different animal feed products can vary depending on the type of animal to which the animal feed product is to be fed and the growth stage of the animal. There are some feed examples that are typical for specific animals at different stages of growth. For example, reference is made to the Veterinary Manual for the Nutritional Requirements of Poultry of the Merck Veterinary Manual by Klasing, 2018, the disclosure which is hereby incorporated by reference in its entirety. The nutritional requirements vary for poultry depending on stage of growth and whether the feed product is for a hen, broiler, turkey, pheasant, bobwhite quail, Peking duck, goose, chicken or turkey as non-limiting examples. Linoleic acid or lysine may be an ingredient that is highly controlled with other ingredients.

One example of a poultry feed based on guidelines includes different components with a w/w percentage value such as crude protein at a minimum of about 15% to as high as 28% with lysine at a minimum of about 0.60% to as high as 2.0% depending on the type of poultry. A minimum methionine can range from 0.35% to as high as 0.5% and crude fat can range from about 3.0% to about 4.0%. Crude fiber as a maximum can range from about 4.0% to about 6.0% and calcium at a minimum from about 0.90% to as high as 1.10% and sometimes as high as 3.5%. Calcium may range from about 1.15% to as high as 1.6% and sometimes with an egg production at 4.4%. Phosphorus can range from about 0.6% to about 0.75%. Salt may range from about 0.25% to 0.3% and may reach a maximum of about 0.5% in some cases. There may be grains and derivatives, of course, as a major component with different types as described above. Corn could be included ranging from 45% to as high as 60%.

The poultry product as described above may include essential oils such as cinnamaldehyde from cinnamon that is used to improve nutrient absorption and protect the stomach and intestinal wall and carbacol from oregano that may stimulate gut microflora and volatile fatty acids and even capcicum from chili peppers. Pre-biotics may be included and the added beta-glucan such as the whole cell Euglena biomass or lysate could act as a pre-biotic in combination with glucomannans to help the digestive tract. This can be combined with a phytase enzyme and a direct fed microbial (DFM) together with lutein. DFM may help inhibit clostridium perfringens and the presence of necrotic enteritis lesions, but the DFM and any enzymes may not be included in a scratch feed.

Similarly, reference is made to the article for Nutritional Requirements and Related Diseases of Small Animals by Sanderson in the Veterinary Manual of the Merck Manual, 2018, the disclosure which is hereby incorporated by reference in its entirety. This article discusses domestic cats and dogs and their nutritional requirements. Reference is also made to the AAFCO Methods for Substantiating Nutritional Adequacy of Dog and Cat Foods, 2013, the disclosure which is hereby incorporated by reference in its entirety.

The Association of American Feed Control Officials (AAFCO) describes a dog or cat nutrient profile and also discusses feeding trials using AAFCO procedures. There are recommended nutrient levels listed in various profiles that provide practical information for pet food manufacturers. Each nutrient listed in each profile has a minimum level and also some have a maximum level. Some guidelines list these levels at different life stages that may vary in terms of nutritional needs. AAFCO established two nutrient profiles for both dogs and cats, and in an example, one for growth and one for reproduction, which includes growing, pregnant and nursing animals, and one for adult maintenance. There also may be dog and cat nutrient profiles that express nutrient levels on a dry matter or moisture-free basis, which will help considering that some pet foods are canned, containing about 75% to 78% moisture. Other pet foods, such as dry pet food, contains about 10% to 12% moisture. Many of the products may contain more than 26% protein on a dry matter basis to meet the minimum levels for crude protein such as in cat food.

Reference is also made to the Starter Pig Recommendations from Kansas State University Agricultural Experiment Station and Cooperative Extension Service (2007), the disclosure which is hereby incorporated by reference in its entirety. The dietary formulations for the animal feed product can vary depending on the size and transition in different phases for a pig. Strategic use may be made of a soybean meal and the importance of lysine with other amino acids. It is possible to use a high amino acid fortification, including additions of spray-dried animal plasma, fish meal, dried whey, whey protein concentrate, spray-dried blood meal, soybean meal, and further processed wood soy products. As the pigs get older, different phased diets may be used, including corn-soybean meal-based dried whey or other source of lactose and as phase increases formulated with high levels of amino acids.

Some protein sources may be added as a supplement such as from fish meal, but usually depends upon a maximum of about 5% because of palatability issues. Dry whey is commonly used and has a higher content of lysine and is lower in salt. In an example, a pig diet could contain anywhere from 25% corn to as high as 65% to 70% corn, and in one example, could range from 50% to 60% w/w. SBM (soybean meal) could range from about a low of 8% to as high as 35% and have a preferred range of about 23% to about 31%. Soy protein for younger pigs could be about 3% and dried whey could be added from about 20% to 30% for a starter transition pig and could be lowered to about 10% to 20% with an average of about 15% and for heavier pigs around 5%. Plasma proteins could be about 6% for starter pigs and oat groats of about 10% to 15%. Fish meal could be added in a range of about 3% to 6% and an average of about 4% to 5%. Overall, the protein for this diet could range on the average from about 18% to 25% and typically about 19% to 20% with lysine ranging from about 1% to 2% and on average about 1.25%. Added calcium and phosphorus could be about 0.7% to 1.0% for calcium and phosphorus of about 0.60% to 0.90% with an average of about 0.7% for phosphorus and 0.8% for calcium in non-limiting examples. Antibiotics could be included.

Reference is also made the Animal Nutrition Handbook, Second Revision, 2009, by Chiba, the disclosure which is hereby incorporated by reference in its entirety, which includes many recommendations for animal feed products for different animals. Section 11 includes pig nutrition and feeding guidelines and shows protein sources that may be used such as a fish meal and dried whey with alternative grains such as oats, barley and wheat, and different fats and oils. One additional ingredient could be medium-chain fatty acids containing 8 to 14 carbons such as coconut oil. Organic acids and probiotics could be added with various enzymes. Section 17 discloses fish, dog and cat nutrition and feeding. Section 12 discloses poultry nutrition and feeding showing a high use of yellow corn in one example such as ranging from 45% to as high as 60% and soybean meal ranging from about 20% to about 40% with other meat and bone meal or meat meal.

These feed product examples show the range of ingredients that could be used in an animal feed product that can be supplemented with the whole cell Euglena biomass or Euglena lysate or their combination with the lower ranges as described above as compared to many other commercially available sources that incorporate much higher ranges of algae, which may include Euglena algae meal at very high percentages. These higher percentages could also have problems with palatability to the animal. An animal feed supplemented with the higher amounts of algae could also increase costs.

This application is related to copending patent application entitled, “EUGLENA DERIVED COMPOSITION HAVING IMMUNE RESPONSE INDUCING COMPONENTS,” and “EUGLENA DERIVED COMPOSITION HAVING BIOMASS AND IMMUNE RESPONSE INDUCING COMPONENTS,” which are filed on the same date and by the same assignee and inventors, the disclosures which are hereby incorporated by reference.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. An animal feed composition, comprising:
a feed component comprising one or more of corn, soy, corn or soy derivatives or byproducts, or grains;
an added whole cell Euglena biomass, including beta-1,3-glucan comprising at least about 90 percent linear, unbranched beta-1,3-glucan polysaccharide polymers having an average molecular weight of about 1.2 to 580 kilodaltons (kDa) and beta-glucan polymer chains having a polymer length of about 7.0 to 3,400 glucose monomers, wherein the whole cell Euglena biomass comprises at least 30 percent beta-1,3-glucan and includes residual media remaining from a heterotrophic fermentation process that produced the whole cell Euglena biomass, wherein the added whole cell Euglena biomass and residual media are about 0.0001 to 0.0124 percent w/w of the composition.

2. The composition according to claim 1, wherein the added whole cell Euglena biomass and residual media are about 0.001 to 0.01 percent w/w of the composition.

3. The composition according to claim 1, where the composition further comprises an added immune response inducing component comprising one or more of vitamin C, Echinacea, aloe, golden seal, ginseng, garlic, bell peppers, ginger, tumeric, gingko biloba, cat's claw, ganoderma, astragalus, humic or fulvic acids, resveratrol or other polyphenols, broccoli, spinach, yogurt, almonds, honey, green tea, papaya, kiwi, poultry, shrimp, sunflower, vitamin D, mushrooms, pumpkin, cinnamon, parsnips, grapes, sweet potatoes, milk, orange juice, rice, carotenoids, figs, glutamine, arginine, an omega-3 fatty acid, vitamin A, vitamin E, selenium, zinc, or a probiotic.

4. The composition according to claim 1, wherein the one or more of corn, soy, corn or soy derivatives or byproducts, or grains comprises about 40% to 95% w/w of the composition.

5. The composition according to claim 1, wherein the feed component further comprises protein in an amount of about 15% to 30% w/w of the composition.

6. The composition according to claim 1, wherein the feed component further comprises lysine in an amount of about 0.60% to 2.0% w/w of the composition.

7. The composition according to claim 1, wherein the residual media remaining from a heterotrophic fermentation process comprises about 10 percent of an initial fermentation concentration.

8. An animal feed composition, comprising:
a feed component comprising one or more of corn, soy, corn or soy derivatives or byproducts, or grains;
an added Euglena biomass lysate having an average particle size of about 2.0 to 500 micrometers and comprising cellular components, including beta-1,3-glucan comprising at least about 90 percent linear, unbranched beta-1,3-glucan polysaccharide polymers having an average molecular weight of about 1.2 to 580 kilodaltons (kDa) and beta-glucan polymer chains having a polymer length of about 7.0 to 3,400 glucose monomers, wherein the Euglena lysate comprises at least 30 percent beta-1,3-glucan and includes residual media remaining from a heterotrophic fermentation process that produced a Euglena biomass and the Euglena lysate, wherein the added Euglena lysate and residual media are about 0.0001 to 0.0124 percent w/w of the composition.

9. The composition according to claim 8, wherein the added Euglena lysate and residual media are about 0.001 to 0.01 percent w/w of the composition.

10. The composition according to claim 8, wherein the composition further comprises an added immune response inducing component comprising one or more of vitamin C, Echinacea, aloe, golden seal, ginseng, garlic, bell peppers, ginger, tumeric, gingko biloba, cat's claw, ganoderma, astragalus, humic or fulvic acids, resveratrol or other polyphenols, broccoli, spinach, yogurt, almonds, honey, green tea, papaya, kiwi, poultry, shrimp, sunflower, vitamin D, mushrooms, pumpkin, cinnamon, parsnips, grapes, sweet potatoes, milk, orange juice, rice, carotenoids, figs, glutamine, arginine, an omega-3 fatty acid, vitamin A, vitamin E, selenium, zinc, or a probiotic.

11. The composition according to claim 8, wherein the one or more of corn, soy, corn or soy derivatives or byproducts, or grains comprises about 40% to 95% w/w of the composition.

12. The composition according to claim 8, wherein the feed component further comprises protein in an amount of about 15% to 30% w/w of the composition.

13. The composition according to claim 8, wherein the feed component further comprises lysine in an amount of about 0.60% to 2.0% w/w of the composition.

14. The composition according to claim 8, wherein the residual media remaining from a heterotrophic fermentation process comprises about 10 percent of an initial fermentation concentration.

15. A method for increasing immunity levels in an animal, comprising administering to the animal an animal feed composition comprising a feed component comprising one or more of corn, soy, corn or soy derivatives or byproducts, or grains and an added whole cell Euglena biomass, including beta-1,3-glucan comprising at least about 90 percent linear, unbranched beta-1,3-glucan polysaccharide polymers having an average molecular weight of about 1.2 to 580 kilodaltons (kDa) and beta-glucan polymer chains having a polymer length of about 7.0 to 3,400 glucose monomers, wherein the whole cell Euglena biomass comprises at least 30 percent beta-1,3-glucan and includes residual media remaining from a heterotrophic fermentation process that produced the whole cell Euglena biomass, wherein the added whole cell Euglena biomass and residual media are about 0.0001 to 0.0124 percent w/w of the composition.

16. The method according to claim 15, wherein the added whole cell Euglena biomass and residual media are about 0.001 to 0.01 percent w/w of the composition.

17. The method according to claim 15, wherein the composition further comprises an added immune response inducing component comprising one or more of vitamin C, Echinacea, aloe, golden seal, ginseng, garlic, bell peppers, ginger, tumeric, gingko biloba, cat's claw, ganoderma, astragalus, humic or fulvic acids, resveratrol or other polyphenols, broccoli, spinach, yogurt, almonds, honey, green tea, papaya, kiwi, poultry, shrimp, sunflower, vitamin D, mushrooms, pumpkin, cinnamon, parsnips, grapes, sweet potatoes, milk, orange juice, rice, carotenoids, figs, glutamine, arginine, an omega-3 fatty acid, vitamin A, vitamin E, selenium, zinc, or a probiotic.

18. The method according to claim 15, wherein the one or more of corn, soy, corn or soy derivatives or byproducts, or grains comprises about 40% to 95% w/w of the composition.

19. The method according to claim 15, wherein the feed component further comprises protein in an amount of about 15% to 30% w/w of the composition.

20. The method according to claim 15, wherein the feed component further comprises lysine in an amount of about 0.60% to 2.0% w/w of the composition.

21. The method according to claim 15, wherein the residual media remaining from a heterotrophic fermentation process comprises about 10 percent of an initial fermentation concentration.

22. A method for increasing immunity levels in an animal, comprising administering to the animal an animal feed composition comprising a feed component comprising one or more of corn, soy, corn or soy derivatives or byproducts, or grains and an added Euglena biomass lysate having an average particle size of about 2.0 to 500 micrometers and comprising cellular components, including beta-1,3-glucan comprising at least about 90 percent linear, unbranched beta-1,3-glucan polysaccharide polymers having an average molecular weight of about 1.2 to 580 kilodaltons (kDa) and beta-glucan polymer chains having a polymer length of about 7.0 to 3,400 glucose monomers, wherein the Euglena lysate comprises at least 30 percent beta-1,3-glucan and includes residual media remaining from a heterotrophic fermentation process that produced a Euglena biomass and the Euglena lysate, wherein the added Euglena lysate and residual media are about 0.0001 to 0.0124 percent w/w of the composition.

23. The method according to claim 22, wherein the added Euglena lysate and residual media are about 0.001 to 0.01 percent w/w of the composition.

24. The method according to claim 22, wherein the composition further comprises an added immune response inducing component comprising one or more of vitamin C, Echinacea, aloe, golden seal, ginseng, garlic, bell peppers, ginger, tumeric, gingko biloba, cat's claw, ganoderma, astragalus, humic or fulvic acids, resveratrol or other polyphenols, broccoli, spinach, yogurt, almonds, honey, green tea, papaya, kiwi, poultry, shrimp, sunflower, vitamin D, mushrooms, pumpkin, cinnamon, parsnips, grapes, sweet potatoes, milk, orange juice, rice, carotenoids, figs, glutamine, arginine, an omega-3 fatty acid, vitamin A, vitamin E, selenium, zinc, or a probiotic.

25. The method according to claim 22, wherein the one or more of corn, soy, corn or soy derivatives or byproducts, or grains comprises about 40% to 95% w/w of the composition.

26. The method according to claim 15, wherein the feed component further comprises protein in an amount of about 15% to 30% w/w of the composition.

27. The method according to claim 15, wherein the feed component further comprises lysine in an amount of about 0.60% to 2.0% w/w of the composition.

28. The method according to claim 15, wherein the residual media remaining from a heterotrophic fermentation process comprises about 10 percent of an initial fermentation concentration.
